(19) United States
(12) Patent Application Publication
(10) Pub. No.: US 2022/0348977 A1
(43) Pub. Date: Nov. 3, 2022

(54) GLYCOPROTEIN MANUFACTURING PROCESS

(71) Applicant: Alexion Pharmaceuticals, Inc., Boston, MA (US)

(72) Inventors: Krishanu MATHUR, Southbury, CT (US); Siguang SUI, South Glastonbury, CT (US)

(21) Appl. No.: 16/490,760

(22) PCT Filed: Mar. 5, 2018
(86) PCT No.: PCT/US2018/020859

§ 371 Date: Sep. 3, 2019

Related U.S. Application Data
(60) Provisional application No. 62/469,190, filed on Mar. 9, 2017.

(51) Int. Cl. C12P 21/00; A61K 38/46; C12N 9/16

(52) U.S. Cl. CPC C12P 21/005; A61K 38/465; C12N 9/16; C12Y 301/03001; C07K 2319/30; C07K 2319/33; C12N 2510/02

(57) ABSTRACT

The disclosure provides a method of producing recombinant alkaline phosphatase comprising: (i) inoculating Chinese Hamster Ovary (CHO) cells expressing recombinant alkaline phosphatase in culture medium; (ii) culturing the CHO cells in the culture medium at a temperature of about 37° C.; (iii) adding a combination of nutrient supplements to the cell culture of (ii) at least one day after inoculation, the combination comprising (a) a first animal-derived component-free (ADCF) nutrient supplement comprising one or more amino acids, vitamins, salts, trace elements, poloxamer and glucose, wherein the first ADCF nutrient supplement does not comprise hypoxanthine, thymidine, insulin, L-glutamine, growth factors, peptides, proteins, hydrolysates, phenol red and 2-mercaptoethanol; and (b) a second ADCF nutrient supplement comprising one or more amino acids, wherein the second ADCF nutrient supplement lacks hypoxanthine, thymidine, insulin, L-glutamine, growth factors, peptides, proteins, hydrolysates, phenol red, 2-mercaptoethanol and poloxamer; (iv) decreasing the temperature of the cell culture of (iii) to about 30° C. about 80 hours to 120 hours after the inoculation; and (v) isolating the recombinant alkaline phosphatase from the cell culture of (iv) by at least one chromatography step.

### FIELD OF THE DISCLOSURE

The disclosure is directed to a method of producing recombinant alkaline phosphatase comprising: (i) inoculating Chinese Hamster Ovary (CHO) cells expressing recombinant alkaline phosphatase in culture medium; (ii) culturing the CHO cells in the culture medium; (iii) adding a combination of nutrient supplements to the cell culture of (ii) at least one day after inoculation, the combination comprising (a) a first animal-derived component-free (ADCF) nutrient supplement comprising one or more amino acids, vitamins, salts, trace elements, poloxamer and glucose, wherein the first ADCF nutrient supplement does not comprise hypoxanthine, thymidine, insulin, L-glutamine, growth factors, peptides, proteins, hydrolysates, phenol red and 2-mercaptoethanol; and (b) a second ADCF nutrient supplement comprising one or more amino acids, wherein the second ADCF nutrient supplement lacks hypoxanthine, thymidine, insulin, L-glutamine, growth factors, peptides, proteins, hydrolysates, phenol red, 2-mercaptoethanol and poloxamer; (iv) decreasing the temperature of the cell culture of (iii) to about 30° C. about 80 to 120 hours after the inoculation; and (v) isolating the recombinant alkaline phosphatase from the cell culture of (iv) by at least one chromatography step.

### BACKGROUND

Hypophosphatasia (HPP) is a life-threatening, genetic, and ultra-rare metabolic disorder that results in a failure to produce functional tissue nonspecific alkaline phosphatase (TNSALP). It leads to the accumulation of unmineralized bone matrix (e.g. rickets, osteomalacia), characterized by hypo-mineralization of bones and teeth. When growing bone does not mineralize properly, impairment of growth disfigures joints and bones. This result in turn impacts motor performance, respiratory function, and may even lead to death. Different forms of HPP include perinatal, infantile, juvenile (or childhood), and adult HPP. Recently, six clinical forms have been defined, most based upon age at symptom onset, including perinatal, benign prenatal, infantile, juvenile, adult, and odonto-HPP. Asfotase alfa is an investigational, first-in-class targeted enzyme replacement therapy designed to address defective endogenous TNSALP levels. For treating HPP with TNSALP, see Whyte et al., 2012 N Engl J Med. 366:904-13.

Asfotase alfa (STRENSIQ®, Alexion Pharmaceuticals, Inc.) is a soluble fusion glycoprotein comprised of the catalytic domain of human TNSALP, a human immunoglobulin G1 Fc domain and a deca-aspartate peptide (i.e., D₁₀) used as a bone-targeting domain. In vitro, asfotase alfa binds with a greater affinity to hydroxyapatite than does soluble TNSALP lacking the deca-aspartate peptide thus allowing the TNSALP moiety of asfotase alfa to efficiently degrade excess local inorganic pyrophosphate (PPi) and restore normal mineralization. Pyrophosphate hydrolysis promotes bone mineralization and its effects are similar among the species evaluated in nonclinical studies. Efficacy studies were conducted in a mouse model of HPP (Akp2^{-/-} mice). The Akp2^{-/-} mouse model, created by inactivating the TNSALP gene (Narisawa et al., 1997 Dev Dyn. 208:432-46), shares many common features of the human condition, including accumulation of unmineralized bone matrix.

### BRIEF SUMMARY

Disclosed herein are improved manufacturing processes that can be used to increase efficiency in the production of alkaline phosphatases (e.g., asfotase alfa). Methods as described here can also be used for maintaining, preserving, modulating and/or improving the enzymatic activity of a recombinant protein, such as alkaline phosphatases (e.g., asfotase alfa) produced by cultured Chinese Hamster Ovary (CHO) cells. Such alkaline phosphatases (e.g., asfotase alfa) are suited for use in therapy, for example, for treatment of conditions associated with decreased alkaline phosphatase protein levels and/or function (e.g., insufficient cleavage of inorganic pyrophosphate (PPi)) in a subject, for example, a human subject.

In one aspect, the present disclosure provides a method for producing a recombinant polypeptide having alkaline phosphatase function. In various embodiments, the alkaline phosphatase function may include any functions of alkaline phosphatase known in the art, such as enzymatic activity toward natural substrates including phosphoethanolamine (PEA), inorganic pyrophosphate (PPi) and pyridoxal 5′-phosphate (PLP). Such recombinant polypeptide can comprise asfotase alpha (SEQ ID NO:1).

In some embodiments, the present disclosure provides a method of producing recombinant alkaline phosphatase comprising: (i) inoculating Chinese Hamster Ovary (CHO) cells expressing recombinant alkaline phosphatase in culture medium; (ii) culturing the CHO cells in the culture medium at a temperature of about 37° C.; (iii) adding a combination of nutrient supplements to the cell culture of (ii) at least one day after inoculation, the combination comprising (a) a first animal-derived component-free (ADCF) nutrient supplement comprising one or more amino acids, vitamins, salts, trace elements, poloxamer and glucose, wherein the first ADCF nutrient supplement does not comprise hypoxanthine, thymidine, insulin, L-glutamine, growth factors, peptides, proteins, hydrolysates, phenol red and 2-mercaptoethanol; and (b) a second ADCF nutrient supplement comprising one or more amino acids, wherein the second ADCF nutrient supplement lacks hypoxanthine, thymidine, insulin, L-glutamine, growth factors, peptides, proteins, hydrolysates, phenol red, 2-mercaptoethanol and poloxamer; (iv) decreasing the temperature of the cell culture of (iii) to about 30° C. about 80 hours to 120 hours after the inoculation; and (v) isolating the recombinant alkaline phosphatase from the cell culture of (iv) by at least one chromatography step.

In some embodiments, (v) occurs 14 days after inoculation. In some embodiments (v) occurs 10 days after inoculation.

In some embodiments, the present disclosure provides a culture medium selected from the group consisting of EX-CELL® 302 Serum-Free Medium; CD DG44 Medium; BD Select™ Medium; SFM4CHO Medium, or a combination thereof.

In some embodiments, the present disclosure provides a culture medium comprising a combination of SFM4CHO Medium and BD Select™ Medium. In some embodiments, the culture medium comprises a combination of SFM4CHO Medium and BD Select™ Medium at a ratio selected from 90/10, 80/20, 75/25, 70/30, 60/40, or 50/50.

In some embodiments, the combination of nutrient supplements are added in a bolus. In some embodiments, the combination of nutrient supplements are added over a period of time ranging from 1 minute to 2 hours. In some embodiments, the combination of nutrient supplements are added 1 to 3 days after inoculation. In some embodiments, the combination of nutrient supplements is added at more than 2 different times. In some embodiments, the combination of nutrient supplements is added at 2 to 6 different times. In some embodiments, the combination of nutrient supplements is added at 4 different times. In some embodiments, the combination of nutrient supplements is added 1 to 3 days after inoculation and 3 to 5 days after inoculation. In some embodiments, the combination of nutrient supplements is added 1 to 3 days after inoculation and 3 to 5 days after inoculation, 5 to 7 days after inoculation, and 7 to 9 days after inoculation. In some embodiments, the combination of nutrient supplements is added about 2 days after inoculation and about 4 days after inoculation, about 6 days after inoculation, and about 8 days after inoculation.

In some embodiments, the present disclosure provides wherein each addition of the first nutrient supplement is added at a concentration of 0.5% to 4% (w/v) of the culture medium. In some embodiments, each addition of the first nutrient supplement is added at a concentration of 2% (w/v) of the culture medium. In some embodiments, each addition of the second nutrient supplement is added at a concentration of 0.05% to 0.8% (w/v) of the culture medium. In some embodiments, each addition of the second nutrient supplement is added at a concentration of 0.2% (w/v) of the culture medium. In some embodiments, the total addition of the first nutrient supplement is added at a concentration of 5% to 20% (w/v) of the culture medium. In some embodiments, the total addition of the first nutrient supplement is added at a concentration of 12% (w/v) of the culture medium. In some embodiments, the total addition of the second nutrient supplement is added at a concentration of 0.5% to 2% (w/v) of the culture medium. In some embodiments, the total addition of the second nutrient supplement is added at a concentration of 1.2% (w/v) of the culture medium.

In some embodiments, the present disclosure provides wherein the first nutrient supplement is CELL BOOST™ 7a, and the second nutrient supplement is CELL BOOST™ 7b (GE Healthcare).

In some embodiments, the present disclosure provides wherein the temperature decrease of (iv) is about 80 hours to 150 hours, or about 90 hours to 100 hours after the inoculation. In some embodiments, the temperature decrease of (iv) is about 96 hours after the inoculation.

In some embodiments, the present disclosure comprises providing zinc concentration (Zn²⁺) from about 20 μM to about 200 μM. In some embodiments, the present disclosure comprises providing zinc concentration in the culture medium to at least about 30 μM. In some embodiments, the method comprises providing zinc concentration to at least about 50 μM. In some embodiments, the method comprises providing zinc concentration to at least about 60 μM. In some embodiments, the method comprises providing zinc concentration in the culture medium to at least about 90 μM. In some embodiments, the method comprises providing zinc concentration to from about 20 μM to about 60 μM. In some embodiments, the method comprises providing zinc concentration to at least about 150 μM. In some embodiments, the method comprises providing zinc concentration to at least about 200 μM.

In some embodiments, the present disclosure provides a chromatography step, wherein the chromatography step comprises at least one of harvest clarification, ultrafiltration, diafiltration, viral inactivation, affinity capture, and combinations thereof.

In some embodiments, the present disclosure provides a method further comprising measuring recombinant alkaline phosphatase activity. In some embodiments, the activity is selected from a method selected from at least one of a pNPP-based alkaline phosphatase enzymatic assay and an inorganic pyrophosphate (PPi) hydrolysis assay. In some embodiments, at least one of the recombinant alkaline phosphatase K_{cat} and Kₘ values increases in an inorganic pyrophosphate (PPi) hydrolysis assay.

In some embodiments, the present disclosure provides a method further comprising determining an integral of viable cell concentration (IVCC). In some embodiments, the IVCC is increased by from about 3.0-fold to about 6.5-fold compared to the method in the absence of steps (iii) and (iv).

In some embodiments, the present disclosure provides a recombinant alkaline phosphatase, wherein the recombinant alkaline phosphatase comprises the structure of W-sALP-X-Fc-Y-Dₙ-Z, wherein

W is absent or is an amino acid sequence of at least one amino acid;

X is absent or is an amino acid sequence of at least one amino acid;

Y is absent or is an amino acid sequence of at least one amino acid;

Z is absent or is an amino acid sequence of at least one amino acid;

Fc is a fragment crystallizable region;

Dₙ is a poly-aspartate, poly-glutamate, or combination thereof, wherein n=10 or 16; and

said sALP is a soluble alkaline phosphatase.

In some embodiments, the sALP comprises an active anchored form of alkaline phosphatase (ALP) without C-terminal glycolipid anchor (GPI).

In some embodiments, the alkaline phosphatase (ALP) is tissue-non-specific alkaline phosphatase (TNALP). In some embodiments, the sALP is encoded by a polynucleotide encoding a polypeptide comprising the sequence as set forth in L1-S485 of SEQ ID NO:1. In some embodiments, the sALP comprises the sequence as set forth in L1-S485 of SEQ ID NO:1. In some embodiments, the sALP is capable of catalyzing the cleavage of inorganic pyrophosphate (PPi). In some embodiments, n=10. In some embodiments, W and Z are absent from said polypeptide. In some embodiments, Fc comprises a CH2 domain, a CH3 domain and a hinge region. In some embodiments, Fc is a constant domain of an immunoglobulin selected from the group consisting of IgG-1, IgG-2, IgG-3, IgG-3 and IgG-4. In some embodiments, Fc is a constant domain of an immunoglobulin IgG-1. In some embodiments, Fc comprises the sequence as set forth in D488-K714 of SEQ ID NO:1. In some embodiments, the recombinant alkaline phosphatase is encoded by a polynucleotide encoding a polypeptide comprising the sequence as set forth in SEQ ID NO:1. In some embodiments, the recombinant alkaline phosphatase is encoded by a first polynucleotide which hybridizes under high stringency conditions to a second polynucleotide comparing the sequence completely complementary to a third polynucleotide encoding a polypeptide comprising the sequence as set forth in SEQ ID NO:1, wherein said high stringency conditions comprise: pre-hybridization and hybridization in 6×SSC, 5× Denhardt's reagent, 0.5% SDS and 100 mg/ml of denatured fragmented salmon sperm DNA at 68° C.; and washes in 2×SSC and 0.5% SDS at room temperature for 10 minutes; in 2×SSC and 0.1% SDS at room temperature for 10 minutes; and in 0.1×SSC and 0.5% SDS at 65° C. three times for 5 minutes. In some embodiments, the recombinant alkaline phosphatase comprises at least 90% sequence identity to SEQ ID NO:1. In some embodiments, the recombinant alkaline phosphatase comprises at least 95% sequence identity to SEQ ID NO:1. In some embodiments, the recombinant alkaline phosphatase as described herein comprises the sequence as set forth in SEQ ID NO:1. In some embodiments, the recombinant alkaline phosphatase as described herein comprises the sequence as set forth in SEQ ID NO:1 and is a dimer thereof.

In some embodiments, the disclosure provides a method of producing recombinant alkaline phosphatase comprising: (i) inoculating Chinese Hamster Ovary (CHO) cells expressing recombinant alkaline phosphatase in culture medium; (ii) culturing the CHO cells in the culture medium at a temperature of from about 36° C. to about 38° C.; from about 36.5° C. to about 37.5° C.; particularly about 37° C.; (iii) adding at least one nutrient supplement to the cell culture, and adding from about 20 μM to about 200 μM Zn²⁺, particularly from about 30 μM Zn²⁺ to 100 μM Zn²⁺; particularly about 90 μM Zn²⁺; (iv) decreasing the temperature of the cell culture of (iii) to about 30° C. about 80 hours to about 120 hours after the inoculation; and (v) isolating the recombinant alkaline phosphatase from the cell culture of (iv) by at least one chromatography step. In some embodiments, the recombinant alkaline phosphatase comprises asfotase alfa.

### BRIEF DESCRIPTION OF THE DRAWINGS

In the appended drawings:

FIG. 1 provides the results of productivity and activity analysis of data from fed shake flasks while varying culture media, addition of a nutrient supplement, and varying zinc concentrations at various end timepoints.

FIGS. 2a and 2b represent the viable cell density (VCD) and cell viability of cultures grown with the addition of various nutrient supplements, and with and without a temperature shift.

FIGS. 3a and 3b represent the specific productivity and ProA Bindable Titer of cultures grown with the addition of various nutrient supplements, and with and without a temperature shift. Data shown for Control Process and Cell Boost 2+5 is the average of two replicates.

FIGS. 4a and 4b represent the specific activity and total activity of cultures grown with the addition of various nutrient supplements, and with and without a temperature shift. Data shown for Control Process and Cell Boost 2+5 is the average of two replicates.

FIG. 5 represents the product volumetric activity profile of cells grown with the addition of various nutrient supplements, and with and without a temperature shift. Feed 1=Cell Boost 2+5; Feed 2=Cell Boost 6; Feed 3=Cell Boost 7a+7b. Data shown for Control Process and Cell Boost 2+5 is the average of two replicates.

FIG. 6 represents the product specific activity profile of cells grown with the addition of various nutrient supplements, and with and without a temperature shift. Feed 1=Cell Boost 2+5; Feed 2=Cell Boost 6; Feed 3=Cell Boost 7a+7b. Data shown for Control Process and Cell Boost 2+5 is the average of two replicates.

FIG. 7 represents the protein impurities of cells grown with the addition of various nutrient supplements, with and without a temperature shift. Brx-1=Control Process with temperature shift; Brx-2=Control Process with temperature shift; Brx-3=Cell Boost 2+5 with temperature shift; Brx-4=Cell Boost 2+5 with temperature shift; Brx-5=Cell Boost 6 with temperature shift; Brx-6=Cell Boost 6 without temperature shift; Brx-7=Cell Boost 7a+7b with temperature shift; Brx-8=Cell Boost 7a+7b without temperature shift. Day 14 values shown.

FIG. 8 represents protein sialylation of cells grown with the addition of various nutrient supplements, and with and without a temperature shift. Brx-1=Control Process with temperature shift; Brx-2=Control Process with temperature shift; Brx-3=Cell Boost 2+5 with temperature shift; Brx-4=Cell Boost 2+5 with temperature shift; Brx-5=Cell Boost 6 with temperature shift; Brx-6=Cell Boost 6 without temperature shift; Brx-7=Cell Boost 7a+7b with temperature shift; Brx-8=Cell Boost 7a+7b without temperature shift. Day 14 values shown.

### DETAILED DESCRIPTION

### Definitions

“About”, “Approximately”: As used herein, the terms “about” and “approximately”, as applied to one or more particular cell culture conditions, refer to a range of values that are similar to the stated reference value for that culture condition or conditions. In certain embodiments, the term “about” refers to a range of values that fall within 25, 20, 19, 18, 17, 16, 15, 14, 13, 12, 11, 10, 9, 8, 7, 6, 5, 4, 3, 2, 1 percent or less of the stated reference value for that culture condition or conditions.

“Amino acid”: The term “amino acid,” as used herein, refers to any of the twenty naturally occurring amino acids that are normally used in the formation of polypeptides, or analogs or derivatives of those amino acids. Amino acids of the present disclosure can be provided in medium to cell cultures. The amino acids provided in the medium may be provided as salts or in hydrate form.

“Culture” and “cell culture”: These terms, as used herein, refer to a cell population that is suspended in a medium (see definition of “medium” below) under conditions suitable for survival and/or growth of the cell population. As will be clear to those of ordinary skill in the art, these terms as used herein may refer to the combination comprising the cell population and the medium in which the population is suspended.

“Batch culture”: The term “batch culture,” as used herein, refers to a method of culturing cells in which all of the components that will ultimately be used in culturing the cells, including the medium (see definition of “medium” below) as well as the cells themselves, are provided at the beginning of the culturing process. A batch culture is typically stopped at some point and the cells and/or components in the medium are harvested and optionally purified. In some embodiments, the methods described here are used in a batch culture.

“Bioreactor”: The term “bioreactor” as used herein refers to any vessel used for the growth of a cell culture (e.g., a mammalian cell culture). The bioreactor can be of any size so long as it is useful for the culturing of cells. Typically, the bioreactor will be at least 1 liter and may be 10, 100, 250, 500, 1000, 2500, 5000, 8000, 10,000, 12,0000, 20,000 liters or more, or any volume in between. The internal conditions of the bioreactor, including, but not limited to pH and temperature, are typically controlled during the culturing period. The bioreactor can be composed of any material that is suitable for holding mammalian or other cell cultures suspended in media under the culture conditions of the present disclosure, including glass, plastic or metal. The term “production bioreactor” as used herein refers to the final bioreactor used in the production of the polypeptide or protein of interest. The volume of the large-scale cell culture production bioreactor is typically at least 500 liters and may be 1000, 2500, 5000, 8000, 10,000, 12,0000, 20,000 liters or more, or any volume in between. One of ordinary skill in the art will be aware of and will be able to choose suitable bioreactors for use in practicing the present disclosure.

“Cell density”: The term “cell density,” as used herein, refers to the number of cells present in a given volume of medium.

“Cell viability”: The term “cell viability,” as used herein, refers to the ability of cells in culture to survive under a given set of culture conditions or experimental variations. The term as used herein also refers to that portion of cells which are alive at a particular time in relation to the total number of cells, living and dead, in the culture at that time.

“Culture” and “cell culture”: These terms, as used herein, refer to a cell population that is suspended in a medium (see definition of “medium” below) under conditions suitable for survival and/or growth of the cell population. As will be clear to those of ordinary skill in the art, these terms as used herein may refer to the combination comprising the cell population and the medium in which the population is suspended.

“Fed-batch culture”: The term “fed-batch culture,” as used herein, refers to a method of culturing cells in which additional components are provided to the culture at some time subsequent to the beginning of the culture process. The provided components typically comprise nutritional supplements for the cells, which have been depleted during the culturing process. A fed-batch culture is typically stopped at some point and the cells and/or components in the medium are harvested and optionally purified. Fed-batch culture may be performed in the corresponding fed-batch bioreactor. In some embodiments, the method comprises a fed-batch culture.

“Fragment”: The term “fragment,” as used herein, refers to a polypeptide and is defined as any discrete portion of a given polypeptide that is unique to or characteristic of that polypeptide. The term as used herein also refers to any discrete portion of a given polypeptide that retains at least a fraction of the activity of the full-length polypeptide. In some embodiments the fraction of activity retained is at least 10% of the activity of the full-length polypeptide. In various embodiments the fraction of activity retained is at least 20%, 30%, 40%, 50%, 60%, 70%, 80% or 90% of the activity of the full-length polypeptide. In other embodiments the fraction of activity retained is at least 95%, 96%, 97%, 98% or 99% of the activity of the full-length polypeptide. In one embodiment, the fraction of activity retained is 100% of the activity of the full-length polypeptide. The term as used herein also refers to any portion of a given polypeptide that includes at least an established sequence element found in the full-length polypeptide. In some embodiments, the sequence element spans at least 4-5 amino acids of the full-length polypeptide. In some embodiments, the sequence element spans at least about 10, 15, 20, 25, 30, 35, 40, 45, 50 or more amino acids of the full-length polypeptide.

“Integrated Viable Cell Density”: The term “integrated viable cell density,” or “IVCD,” as used herein, refers to the average density of viable cells over the course of the culture multiplied by the amount of time the culture has run. Assuming the amount of polypeptide and/or protein produced is proportional to the number of viable cells present over the course of the culture, integrated viable cell density is a useful tool for estimating the amount of polypeptide and/or protein produced over the course of the culture.

“Medium”, “cell culture medium”, and “culture medium”: These terms, as used herein, refer to a solution containing nutrients which nourish growing mammalian cells. Typically, these solutions provide essential and non-essential amino acids, vitamins, energy sources, lipids, and trace elements required by the cell for minimal growth and/or survival. The solution may also contain components that enhance growth and/or survival above the minimal rate, including hormones and growth factors. The solution is, e.g., formulated to a pH and salt concentration optimal for cell survival and proliferation. In some embodiments, a culture medium may be a “defined media”—a serum-free media that contains no proteins, hydrolysates or components of unknown composition. Defined media are free of animal-derived components and all components have a known chemical structure. In some embodiments, the culture medium is a basal medium, i.e., an undefined medium containing a carbon source, water, salts, a source of amino acids and nitrogen (e.g., animal, e.g., beef, or yeast extracts). Various mediums are commercially available and are known to those in the art. In some embodiments, the culture medium is selected from EX-CELL® 302 Serum-Free Medium (Signam Aldrich, St. Louis, Mo.), CD DG44 Medium (ThermoFisher Scientific, Waltham, Mass.), BD Select Medium (BD Biosciences, San Jose, Calif.), or a mixture thereof. a mixture of BD Select Medium with SFM4CHO Medium (Hyclone, Logan Utah). In some embodiments, the culture medium comprises a combination of SFM4CHO Medium and BD Select™ Medium. In some embodiments, the culture medium comprises a combination of SFM4CHO Medium and BD Select™ Medium at a ratio selected from 90/10, 80/20, 75/25, 70/30, 60/40, or 50/50. In some embodiments, the culture medium comprises a combination of SFM4CHO Medium and BD Select™ Medium at a ratio of 70/30 to 90/10. In some embodiments, the culture medium comprises a combination of SFM4CHO Medium and BD Select™ Medium at a ratio 75/25.

“Metabolic waste product”: The term “metabolic waste product,” as used herein, refers to compound produced by the cell culture as a result of normal or non-normal metabolic processes that are in some way detrimental to the cell culture, particularly in relation to the expression or activity of a desired recombinant polypeptide or protein. For example, the metabolic waste products may be detrimental to the growth or viability of the cell culture, may decrease the amount of recombinant polypeptide or protein produced, may alter the folding, stability, glycosylation or other post-translational modification of the expressed polypeptide or protein, or may be detrimental to the cells and/or expression or activity of the recombinant polypeptide or protein in any number of other ways. Exemplary metabolic waste products include lactate, which is produced as a result of glucose metabolism, and ammonium, which is produced as a result of glutamine metabolism. In one embodiment, methods are taken to slow production of, reduce or even eliminate metabolic waste products in cell cultures.

“Osmolality” and “osmolarity”: Osmolality is a measure of the osmotic pressure of dissolved solute particles in an aqueous solution. The solute particles include both ions and non-ionized molecules. Osmolality is expressed as the concentration of osmotically active particles (i.e., osmoles) dissolved in 1 kg of solution (1 mOsm/kg H₂O at 38° C. is equivalent to an osmotic pressure of 19 mm Hg). “Osmolarity,” by contrast, refers to the number of solute particles dissolved in 1 liter of solution. When used herein the abbreviation “mOsm” means “milliosmoles/kg solution”.

“Perfusion culture”: The term “perfusion culture,” as used herein, refers to a method of culturing cells in which additional components are provided continuously or semi-continuously to the culture subsequent to the beginning of the culture process. The provided components typically comprise nutritional supplements for the cells, which have been depleted during the culturing process. A portion of the cells and/or components in the medium are typically harvested on a continuous or semi-continuous basis and are optionally purified. In some embodiments, the nutritional supplements as described herein are added in a perfusion culture, i.e., they are provided continuously over a defined period of time.

“Polypeptide”: The term “polypeptide,” as used herein, refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond.

“Protein”: The term “protein,” as used herein, refers to one or more polypeptides that function as a discrete unit. If a single polypeptide is the discrete functioning unit and does not require permanent physical association with other polypeptides in order to form the discrete functioning unit, the terms “polypeptide” and “protein” as used herein are used interchangeably.

“Recombinantly-expressed polypeptide” and “recombinant polypeptide”: These terms, as used herein, refer to a polypeptide expressed from a host cell that has been genetically engineered to express that polypeptide. The recombinantly-expressed polypeptide can be identical or similar to a polypeptide that is normally expressed in the mammalian host cell. The recombinantly-expressed polypeptide can also be foreign to the host cell, i.e., heterologous to peptides normally expressed in the host cell. Alternatively, the recombinantly-expressed polypeptide can be chimeric in that portions of the polypeptide contain amino acid sequences that are identical or similar to polypeptides normally expressed in the mammalian host cell, while other portions are foreign to the host cell.

“Seeding”: The term “seeding,” as used herein, refers to the process of providing a cell culture to a bioreactor or another vessel. The cells may have been propagated previously in another bioreactor or vessel. Alternatively, the cells may have been frozen and thawed immediately prior to providing them to the bioreactor or vessel. The term refers to any number of cells, including a single cell. In various embodiments, alkaline phosphatase (e.g., asfotase alfa) is produced by a process in which cells are seeded in a density of about 1.0×10⁵ cells/mL, 1.5×10⁵ cells/mL, 2.0×10⁵ cells/mL, 2.5×10⁵ cells/mL, 3.0×10⁵ cells/mL, 3.5×10⁵ cells/mL, 4.0×10⁵ cells/mL, 4.5×10⁵ cells/mL, 5.0×10⁵ cells/mL, 5.5×10⁵ cells/mL, 6.0×10⁵ cells/mL, 6.5×10⁵ cells/mL, 7.0×10⁵ cells/mL, 7.5×10⁵ cells/mL, 8.0×10⁵ cells/mL, 8.5×10⁵ cells/mL, 9.0×10⁵ cells/mL, 9.5×10⁵ cells/mL, 1.0×10⁶ cells/mL, 1.5×10⁶ cells/mL, 2.0×10⁶ cells/mL, or a higher density. In one particular embodiment, in such process cells are seeded in a density of about 4.0×10⁵ cells/mL, 5.5×10⁵ cells/mL or 8.0×10⁵ cells/mL.

“Titer”: The term “titer,” as used herein, refers to the total amount of recombinantly-expressed polypeptide or protein produced by a cell culture divided by a given amount of medium volume. Titer is typically expressed in units of milligrams of polypeptide or protein per milliliter of medium.

Acronyms used herein include, e.g., VCD: Viable Cell Density; IVCC: Integral of Viable Cell Concentration; TSAC: Total Sialic Acid Content; HPAE-PAD: High-Performance Anion Exchange Chromatography with Pulsed Amperometric Detection; SEC: Size Exclusion Chromatography; AEX: Anion Exchange Chromatography; LoC: Lab-on-Chip; and MALDI-TOF: Matrix Assisted Laser Desorption/Ionization—Time of Flight.

As used herein, the term “hydrophobic interaction chromatography (HIC) column” refers to a column containing a stationary phase or resin and a mobile or solution phase in which the hydrophobic interaction between a protein and hydrophobic groups on the stationary phase or resin separates a protein from impurities including fragments and aggregates of the subject protein, other proteins or protein fragments and other contaminants such as cell debris, or residual impurities from other purification steps. The stationary phase or resin comprises a base matrix or support such as a cross-linked agarose, silica or synthetic copolymer material to which hydrophobic ligands are attached. Examples of such stationary phase or resins include phenyl-, butyl-, octyl-, hexyl- and other alkyl substituted agarose, silica, or other synthetic polymers. Columns may be of any size containing the stationary phase, or may be open and batch processed. In some embodiments, the recombinant alkaline phosphatase is isolated from the cell culture using HIC.

As used herein, the term “preparation” refers to a solution comprising a protein of interest (e.g., a recombinant alkaline phosphatase described herein) and at least one impurity from a cell culture producing such protein of interest and/or a solution used to extract, concentrate, and/or purify such protein of interest from the cell culture. For example, a preparation of a protein of interest (e.g., a recombinant alkaline phosphatase described herein) may be prepared by homogenizing cells, which grow in a cell culture and produce such protein of interest, in a homogenizing solution. In some embodiments, the preparation is then subjected to one or more purification/isolation process, e.g., a chromatography step.

As used herein, the term “solution” refers to a homogeneous, molecular mixture of two or more substances in a liquid form. Specifically, in some embodiments, the proteins to be purified, such as the recombinant alkaline phosphatases or their fusion proteins (e.g., asfotase alfa) in the present disclosure represent one substance in a solution. The term “buffer” or “buffered solution” refers to solutions which resist changes in pH by the action of its conjugate acid-base range. Examples of buffers that control pH at ranges of about pH 5 to about pH 7 include HEPES, citrate, phosphate, and acetate, and other mineral acid or organic acid buffers, and combinations of these. Salt cations include sodium, ammonium, and potassium. As used herein the term “loading buffer/solution” or “equilibrium buffer/solution” refers to the buffer/solution containing the salt or salts which is mixed with the protein preparation for loading the protein preparation onto a chromatography column, e.g., HIC column. This buffer/solution is also used to equilibrate the column before loading, and to wash to column after loading the protein. The “elution buffer/solution” refers to the buffer/solution used to elute the protein from the column. As used herein, the term “solution” refers to either a buffered or a non-buffered solution, including water.

The term “minimize,” or in other similar forms, refers to reducing the concentration of certain molecules (e.g., at least one of metal ions) in certain solutions (e.g., a preparation comprising a recombinant alkaline phosphatase, or other solutions used in the purification processes for such recombinant alkaline phosphatase, such as solutions for a chromatography step, e.g., HIC step), preferably to less than a certain level. For example, the method described herein may comprise minimizing or reducing the concentration of certain metal ions (e.g., Ni, Cu, Co, Mn, etc.) in a preparation comprising a recombinant alkaline phosphatase produced by a cell culture or in solutions for at least one purification processes for such recombinant alkaline phosphatase (e.g., solutions for a chromatography step) to less than a certain level so that such metal ions may not interfere the zinc-enzyme structural formulation for the purified recombinant alkaline phosphatase. Thus, by minimizing the concentration of certain metal ions to less than said certain level, the purified recombinant alkaline phosphatase has increased activity compared to, or does not lose as much activity as, the recombinant alkaline phosphatase purified through same processes but without minimizing the concentration of said certain metal ions.

The present disclosure provides a method of improving the yield and enzymatic function of a recombinant protein which is expressed by cell culture (e.g., mammalian cells including but not limited to Chinese Hamster Ovary (CHO) cells). Specifically, a recombinant protein may be produced by a certain type of cells (e.g., mammalian cells including but not limited to Chinese Hamster Ovary (CHO) cells) through, for example, a fermentation process. The total processes of inoculation and growth of the cells, induction of protein expression, and various parameter optimizations for protein expression are referred as upstream processing steps. Correspondingly, the downstream processing steps may include, e.g., the recovery and purification of the produced proteins (i.e., separation of the produced proteins from other impurities and/or contaminants originated from the cells and the culture medium). Exemplary downstream process steps include, for example, protein capturing from harvest, removing host cell debris, host cell proteins (HCPs), and host cell DNAs, endotoxins, viruses and other containments, buffer-exchanging, and formulation adjustment, etc.

The present disclosure provides a method of improving the enzymatic function of an alkaline phosphatase (e.g., asfotase alfa) which is produced by cell culture.

The present disclosure provides a method of culturing cells (e.g., mammalian cells including but not limited to Chinese Hamster Ovary (CHO) cells) expressing a recombinant protein. The present disclosure provides manufacturing systems for the production of an alkaline phosphatase (e.g., asfotase alfa) by cell culture. In certain embodiments, systems are provided that minimize production of one or more metabolic products that are detrimental to cell growth, viability, and/or protein production or quality. In particular embodiments, the cell culture is a batch culture, a fed-batch culture, a culture or a continuous culture.

### Alkaline Phosphatases (ALPS)

The present disclosure relates to the manufacturing of an alkaline phosphatase protein (e.g., asfotase alfa) in recombinant cell culture. The alkaline phosphatase protein includes any polypeptides or molecules comprising polypeptides that comprise at least some alkaline phosphatase activity. In various embodiments, the alkaline phosphatase disclosed herein includes any polypeptide having alkaline phosphatase functions, which may include any functions of alkaline phosphatase known in the art, such as enzymatic activity toward natural substrates including phosphoethanolamine (PEA), inorganic pyrophosphate (PPi) and pyridoxal 5′-phosphate (PLP).

In certain embodiments, such alkaline phosphatase protein, after being produced and then purified by the methods disclosed herein, can be used to treat or prevent alkaline phosphatase-related diseases or disorders. For example, such alkaline phosphatase protein may be administered to a subject having decreased and/or malfunctioned endogenous alkaline phosphatase, or having overexpressed (e.g., above normal level) alkaline phosphatase substrates. In some embodiments, the alkaline phosphatase protein in this disclosure is a recombinant protein. In some embodiments, the alkaline phosphatase protein is a fusion protein. In some embodiments, the alkaline phosphatase protein in this disclosure specifically targets a cell type, tissue (e.g., connective, muscle, nervous, or epithelial tissues), or organ (e.g., liver, heart, kidney, muscles, bones, cartilage, ligaments, tendons, etc.). For example, such alkaline phosphatase protein may comprise a full-length alkaline phosphatase (ALP) or fragment of at least one alkaline phosphatase (ALP). In some embodiments, the alkaline phosphatase protein comprises a soluble ALP (sALP) linked to a bone-targeting moiety (e.g., a negatively-charged peptide as described below). In some embodiments, the alkaline phosphatase protein comprises a soluble ALP (sALP) linked to an immunoglobulin moiety (full-length or fragment). For example, such immunoglobulin moiety may comprise a fragment crystallizable region (Fc). In some embodiments, the alkaline phosphatase protein comprises a soluble ALP (sALP) linked to both a bone-targeting moiety and an immunoglobulin moiety (full-length or fragment). For more detailed description of the alkaline phosphatase protein disclosed herein, see PCT Publication Nos. WO 2005/103263 and WO 2008/138131, the teachings of both of which are incorporated by reference herein in their entirety.

In some embodiments, the alkaline phosphatase protein described herein comprises any one of the structures selected from the group consisting of: sALP-X, X-sALP, sALP-Y, Y-sALP, sALP-X-Y, sALP-Y-X, X-sALP-Y, X-Y-sALP, Y-sALP-X, and Y-X-sALP, wherein X comprises a bone-targeting moiety, as described herein, and Y comprises an immunoglobulin moiety, as described herein. In one embodiment, the alkaline phosphatase protein comprises the structure of W-sALP-X-Fc-Y-Dₙ/Eₙ-Z, wherein W is absent or is an amino acid sequence of at least one amino acid; X is absent or is an amino acid sequence of at least one amino acid; Y is absent or is an amino acid sequence of at least one amino acid; Z is absent or is an amino acid sequence of at least one amino acid; Fc is a fragment crystallizable region; Dₙ/Eₙ is a polyaspartate, polyglutamate, or combination thereof wherein n=8-20; and sALP is a soluble alkaline phosphatase (ALP). In some embodiments, Dₙ/Eₙ is a polyaspartate sequence. For example, Dₙ may be a polyaspartate sequence wherein n is any number between 8 and 20 (both included) (e.g., n may be 8, 9, 10, 11, 12, 13, 14, 15, 16, 17, 18, 19, and 20). In one embodiment, Dₙ is D₁₀ or D16. In some embodiments, Dₙ/Eₙ is a polyglutamate sequence. For example, Eₙ may be a polyglutamate sequence wherein n is any number between 8 and 20 (both included) (e.g., n may be 8, 9, 10, 11, 12, 13, 14, 15, 16, 17, 18, 19, and 20). In one embodiment, Eₙ is E₁₀ or E₁₆.

For example, such sALPs may be fused to the full-length or fragment (e.g., the fragment crystallizable region (Fc)) of an immunoglobulin molecule. In some embodiments, the recombinant polypeptide comprises a structure of W-sALP-X-Fc-Y-Dₙ-Z, wherein W is absent or is an amino acid sequence of at least one amino acid; X is absent or is an amino acid sequence of at least one amino acid; Y is absent or is an amino acid sequence of at least one amino acid; Z is absent or is an amino acid sequence of at least one amino acid; Fc is a fragment crystallizable region; Dₙ is a poly-aspartate, poly-glutamate, or combination thereof, wherein n=10 or 16; and said sALP is a soluble alkaline phosphatase. In one embodiment, n=10. In another embodiment, W and Z are absent from said polypeptide. In some embodiments, said Fc comprises a CH2 domain, a CH3 domain and a hinge region. In some embodiments, said Fc is a constant domain of an immunoglobulin selected from the group consisting of IgG-1, IgG-2, IgG-3, IgG-3 and IgG-4. In one embodiment, said Fc is a constant domain of an immunoglobulin IgG-1. In one particular embodiment, said Fc comprises the sequence as set forth in D488-K714 of SEQ ID NO:1.

In some embodiments, the alkaline phosphatase disclosed herein comprises the structure of W-sALP-X-Fc-Y-Dₙ-Z, wherein W is absent or is an amino acid sequence of at least one amino acid; X is absent or is an amino acid sequence of at least one amino acid; Y is absent or is an amino acid sequence of at least one amino acid; Z is absent or is an amino acid sequence of at least one amino acid; Fc is a fragment crystallizable region; Dₙ is a poly-aspartate, poly-glutamate, or combination thereof, wherein n=10 or 16; and said sALP is a soluble alkaline phosphatase. Such sALP is capable of catalyzing the cleavage of at least one of phosphoethanolamine (PEA), inorganic pyrophosphate (PPi) and pyridoxal 5′-phosphate (PLP). In various embodiments, the sALP disclosed herein is capable of catalyzing the cleavage of inorganic pyrophosphate (PPi). Such sALP may comprise all amino acids of the active anchored form of alkaline phosphatase (ALP) without C-terminal glycolipid anchor (GPI). Such ALP may be at least one of tissue-non-specific alkaline phosphatase (TNALP), placental alkaline phosphatase (PALP), germ cell alkaline phosphatase (GCALP), and intestinal alkaline phosphatase (IAP), or their chimeric or fusion forms or variants disclosed herein. In one particular embodiment, the ALP comprises tissue-non-specific alkaline phosphatase (TNALP). In another embodiment, the sALP disclosed herein is encoded by a polynucleotide encoding a polypeptide comprising the sequence as set forth in L1-S485 of SEQ ID NO:1. In yet another embodiment, the sALP disclosed herein comprises the sequence as set forth in L1-S485 of SEQ ID NO:1.

In one embodiment, the alkaline phosphatase protein comprises the structure of TNALP-Fc-D₁₀ (SEQ ID NO: 1, as listed below). Underlined asparagine (N) residues correspond to potential glycosylation sites (i.e., N 123, 213, 254, 286, 413 & 564). Bold underlined amino acid residues (L₄₈₆-K₄₈₇ & D₇₁₅-I₇₁₆) correspond to linkers between sALP and Fc, and Fc and D₁₀ domains, respectively.

```
(SEQ ID NO: 1)
                10 20 30 40
LVPEKEKDPK YWRDQAQETL KYALELQKLN TNVAKNVIMF
                50 60 70 80
LGDGMGVSTV TAARILKGQL HHNPGEETRL EMDKFPFVAL
                90 100 110 120
SKTYNTNAQV PDSAGTATAY LCGVKANEGT VGVSAATERS
              130 140 150 160
RCNTTQGNEV TSILRWAKDA GKSVGIVTTT RVNHATPSAA
              170 180 190 200
YAHSADRDWY SDNEMPPEAL SQGCKDIAYQ LMHNIRDIDV
              210 220 230 240
IMGGGRKYMY PKNKTDVEYE SDEKARGTRL DGLDLVDTWK
              250 260 270 280
SFKPRYKHSH FIWNRTELLT LDPHNVDYLL GLFEPGDMQY
              290 300 310 320
ELNRNNVTDP SLSEMVVVAI QILRKNPKGF FLLVEGGRID
              330 340 350 360
HGHHEGKAKQ ALHEAVEMDR AIGQAGSLTS SEDTLTVVTA
              370 380 390 400
DHSHVFTFGG YTPRGNSIFG LAPMLSDTDK KPFTAILYGN
              410 420 430 440
GPGYKVVGGE RENVSMVDYA HNNYQAQSAV PLRHETHGGE
              450 460 470 480
DVAVFSKGPM AHLLHGVHEQ NYVPHVMAYA ACIGANLGHC
              490 500 510 520
APASSLKDKT HTCPPCPAPE LLGGPSVFLF PPKPKDTLMI
              530 540 550 560
SRTPEVTCVV VDVSHEDPEV KFNWYVDGVE VHNAKTKPRE
              570 580 590 600
EQYNSTYRVV SVLTVLHQDW LNGKEYKCKV SNKALPAPIE
              610 620 630 640
KTISKAKGQP REPQVYTLPP SREEMTKNQV SLTCLVKGFY
              650 660 670 680
PSDIAVEWES NGQPENNYKT TPPVLDSDGS FFLYSKLTVD
              690 700 710 720
KSRWQQGNVF SCSVMHEALH NHYTQKSLSL SPGKDIDDDD
DDDDDD
```

Each polypeptide or monomer is composed of five portions. The first portion (sALP) containing amino acids L1-S485 is the soluble part of the human tissue non-specific alkaline phosphatase enzyme, which contains the catalytic function. The second portion contains amino acids L486-K487 as a linker. The third portion (Fc) containing amino acids D488-K714 is the Fc part of the human Immunoglobulin gamma 1 (IgG1) containing hinge, CH₂ and CH₃ domains. The fourth portion contains D715-1716 as a linker. The fifth portion contains amino acids D717-D726 (D₁₀), which is a bone targeting moiety that allows asfotase alfa to bind to the mineral phase of bone. In addition, each polypeptide chain contains six potential glycosylation sites and eleven cysteine (Cys) residues. Cys102 exists as free cysteine. Each polypeptide chain contains four intra-chain disulfide bonds between Cys122 and Cys184, Cys472 and Cys480, Cys528 and Cys588, and Cys634 and Cys692. The two polypeptide chains are connected by two inter-chain disulfide bonds between Cys493 on both chains and between Cys496 on both chains. In addition to these covalent structural features, mammalian alkaline phosphatases are thought to have four metal-binding sites on each polypeptide chain, including two sites for zinc, one site for magnesium and one site for calcium.

There are four known isozymes of ALP, namely tissue non-specific alkaline phosphatase (TNALP) further described below, placental alkaline phosphatase (PALP) (as described e.g., in GenBank Accession Nos. NP_112603 and NP_001623), germ cell alkaline phosphatase (GCALP) (as described, e.g., in GenBank Accession No. P10696) and intestinal alkaline phosphatase (IAP) (as described, e.g., in GenBank Accession No. NP_001622). These enzymes possess very similar three-dimensional structures. Each of their catalytic sites contains four metal-binding domains, for metal ions that are necessary for enzymatic activity, including two Zn and one Mg. These enzymes catalyze the hydrolysis of monoesters of phosphoric acid and also catalyze a transphosphorylation reaction in the presence of high concentrations of phosphate acceptors. Three known natural substrates for ALP (e.g., TNALP) include phosphoethanolamine (PEA), inorganic pyrophosphate (PPi) and pyridoxal 5′-phosphate (PLP) (Whyte et al., 1995 J Clin Invest 95:1440-1445). An alignment between these isozymes is shown in FIG. 30 of WO 2008/138131, the teachings of which are incorporated by reference herein in their entirety.

The alkaline phosphatase protein in this disclosure may comprise a dimer or multimers of any ALP protein, alone or in combination. Chimeric ALP proteins or fusion proteins may also be produced, such as the chimeric ALP protein that is described in Kiffer-Moreira et al. 2014 PLoS One 9:e89374, the entire teachings of which are incorporated by reference herein in its entirety.

In one particular embodiment, the alkaline phosphatase disclosed herein is encoded by a polynucleotide encoding a polypeptide comprising the sequence as set forth in SEQ ID NO:1. In some embodiments, the alkaline phosphatase disclosed herein is encoded by a polynucleotide encoding a polypeptide comprising a sequence having 80%, 85%, 88%, 90%, 91%, 92%, 93%, 94%, 95%, 96%, 97%, 98% or 99% identity to SEQ ID NO:1. In some embodiments, the alkaline phosphatase disclosed herein is encoded by a polynucleotide encoding a polypeptide comprising a sequence having 95% or 99% identity to SEQ ID NO:1. In another embodiment, the alkaline phosphatase disclosed herein comprises the sequence as set forth in SEQ ID NO:1.

### TNALP

As indicated above, TNALP is a membrane-bound protein anchored through a glycolipid to its C-terminus (for human TNALP, see UniProtKB/Swiss-Prot Accession No. P05186). This glycolipid anchor (GPI) is added post translationally after removal of a hydrophobic C-terminal end which serves both as a temporary membrane anchor and as a signal for the addition of the GPI. Hence, in one embodiment a soluble human TNALP comprises a TNALP wherein the first amino acid of the hydrophobic C-terminal sequence, namely alanine, is replaced by a stop codon. The soluble TNALP (herein called sTNALP) so formed contains all amino acids of the native anchored form of TNALP that are necessary for the formation of the catalytic site but lacks the GPI membrane anchor. Known TNALPs include, e.g., human TNALP [GenBank Accession Nos. NP-000469, AAI10910, AAH90861, AAH66116, AAH21289, and AAI26166]; rhesus TNALP [GenBank Accession No. XP-001109717]; rat TNALP [GenBank Accession No. NP_037191]; dog TNALP [GenBank Accession No. AAF64516]; pig TNALP [GenBank Accession No. AAN64273], mouse TNALP [GenBank Accession No. NP_031457], bovine TNALP [GenBank Accession Nos. NP_789828, NP_776412, AAM 8209, and AAC33858], and cat TNALP [GenBank Accession No. NP_001036028].

As used herein, the terminology “extracellular domain” is meant to refer to any functional extracellular portion of the native protein (e.g., without the peptide signal). Recombinant sTNALP polypeptide retaining original amino acids 1 to 501 (18 to 501 when secreted), amino acids 1 to 502 (18 to 502 when secreted), amino acids 1 to 504 (18 to 504 when secreted), or amino acids 1 to 505 (18-505 when secreted) are enzymatically active (see Oda et al., 1999 J. Biochem 126:694-699). This indicates that amino acid residues can be removed from the C-terminal end of the native protein without affecting its enzymatic activity. Furthermore, the soluble human TNALP may comprise one or more amino acid substitutions, wherein such substitution(s) does not reduce or at least does not completely inhibit the enzymatic activity of the sTNALP. For example, certain mutations that are known to cause hypophosphatasia (HPP) are listed in PCT Publication No. WO 2008/138131 and should be avoided to maintain a functional sTNALP.

### Negatively-Charged Peptide

The alkaline phosphatase protein of the present disclosure may comprise a target moiety which may specifically target the alkaline phosphatase protein to a pre-determined cell type, tissue, or organ. In some embodiments, such pre-determined cell type, tissue, or organ is bone tissues. Such bone-targeting moiety may include any known polypeptide, polynucleotide, or small molecule compounds known in the art. For example, negatively-charged peptides may be used as a bone-targeting moiety. In some embodiments, such negatively-charged peptides may be a poly-aspartate, poly-glutamate, or combination thereof (e.g., a polypeptide comprising at least one aspartate and at least one glutamate, such as a negatively-charged peptide comprising a combination of aspartate and glutamate residues). In some embodiments, such negatively-charged peptides may be D₆, D₇, D₈, D₉, D₁₀, D₁₁, D₁₂, D₁₃, D₁₄, D₁₅, D₁₆, D₁₇, D₁₈, D₁₉, D₂₀, or a polyaspartate having more than 20 aspartates. In some embodiments, such negatively-charged peptides may be E₆, E₇, E₈, E₉, E₁₀, E₁₁, E₁₂, E₁₃, E₁₄, E₁₅, Eu₁₆, E₁₇, E₁₈, E₁₉, E₂₀, or a polyglutamate having more than 20 glutamates. In one embodiment, such negatively-charged peptides may comprise at least one selected from the group consisting of D₁₀ to D₁₆ or E₁₀ to Eu₁₆.

### Spacer

In some embodiments, the alkaline phosphatase protein of the present disclosure comprises a spacer sequence between the ALP portion and the targeting moiety portion. In one embodiment, such alkaline phosphatase protein comprises a spacer sequence between the ALP (e.g., TNALP) portion and the negatively-charged peptide targeting moiety. Such spacer may be any polypeptide, polynucleotide, or small molecule compound. In some embodiments, such spacer may comprise fragment crystallizable region (Fc) fragments. Useful Fc fragments include Fc fragments of IgG that comprise the hinge, and the CH₂ and CH₃ domains. Such IgG may be any of IgG-1, IgG-2, IgG-3, IgG-3 and IgG-4, or any combination thereof.

Without being limited to this theory, it is believed that the Fc fragment used in bone-targeted sALP fusion proteins (e.g., asfotase alfa) acts as a spacer, which allows the protein to be more efficiently folded given that the expression of sTNALP-Fc-D₁₀ was higher than that of sTNALP-D₁₀ (see Example 2 below). One possible explanation is that the introduction of the Fc fragment alleviates the repulsive forces caused by the presence of the highly negatively-charged D₁₀ sequence added at the C-terminus of the sALP sequence exemplified herein. In some embodiments, the alkaline phosphatase protein described herein comprises a structure selected from the group consisting of: sALP-Fc-D₁₀, sALP-D₁₀-Fc, D₁₀-sALP-Fc, D₁₀-Fc-sALP, Fc-sALP-D₁₀, and Fc-D₁₀-sALP. In other embodiments, the D₁₀ in the above structures is substituted by other negatively-charged polypeptides (e.g., D₈, D₁₆, E₁₀, E₈, Eu₁₆, etc.).

Useful spacers for the present disclosure include, e.g., polypeptides comprising a Fc, and hydrophilic and flexible polypeptides able to alleviate the repulsive forces caused by the presence of the highly negatively-charged bone-targeting sequence (e.g., D₁₀) added at the C-terminus of the sALP sequence.

### Dimers/Tetramers

In specific embodiments, the bone-targeted sALP fusion proteins of the present disclosure are associated so as to form dimers or tetramers.

In the dimeric configuration, the steric hindrance imposed by the formation of the interchain disulfide bonds is presumably preventing the association of sALP domains to associate into the dimeric minimal catalytically-active protein that is present in normal cells.

Without being limited to this particular theory, it is believed that in its tetrameric structure, the association of the fusion proteins involves one sALP domain from one dimer linking to another sALP domain from a different dimer.

The bone-targeted sALP may further optionally comprise one or more additional amino acids 1) downstream from the negatively-charged peptide (e.g., the bone tag); and/or 2) between the negatively-charged peptide (e.g., the bone tag) and the Fc fragment; and/or 3) between the spacer (e.g., an Fc fragment) and the sALP fragment. This could occur, for example, when the cloning strategy used to produce the bone-targeting conjugate introduces exogenous amino acids in these locations. However the exogenous amino acids should be selected so as not to provide an additional GPI anchoring signal. The likelihood of a designed sequence being cleaved by the transamidase of the host cell can be predicted as described by Ikezawa, 2002 Glycosylphosphatidylino sitol (GPI)-anchored proteins. Biol Pharm Bull. 25:409-17.

The present disclosure also encompasses a fusion protein that is post-translationally modified, such as by glycosylation including those expressly mentioned herein, acetylation, amidation, blockage, formylation, gamma-carboxyglutamic acid hydroxylation, methylation, phosphorylation, pyrrolidone carboxylic acid, and sulfation.

### Asfotase Alfa

Asfotase alfa is a soluble Fc fusion protein consisting of two TNALP-Fc-D₁₀ polypeptides each with 726 amino acids as shown in SEQ ID NO:1. Each polypeptide or monomer is composed of five portions. The first portion (sALP) containing amino acids L1-S485 is the soluble part of the human tissue non-specific alkaline phosphatase enzyme, which contains the catalytic function. The second portion contains amino acids L486-K487 as a linker. The third portion (Fc) containing amino acids D488-K714 is the Fc part of the human Immunoglobulin gamma 1 (IgG1) containing hinge, CH₂ and CH₃ domains. The fourth portion contains D715-1716 as a linker. The fifth portion contains amino acids D717-D726 (D₁₀), which is a bone targeting moiety that allows asfotase alfa to bind to the mineral phase of bone. In addition, each polypeptide chain contains six potential glycosylation sites and eleven cysteine (Cys) residues. Cys102 exists as free cysteine. Each polypeptide chain contains four intra-chain disulfide bonds between Cys122 and Cys184, Cys472 and Cys480, Cys528 and Cys588, and Cys634 and Cys692. The two polypeptide chains are connected by two inter-chain disulfide bonds between Cys493 on both chains and between Cys496 on both chains. In addition to these covalent structural features, mammalian alkaline phosphatases are thought to have four metal-binding sites on each polypeptide chain, including two sites for zinc, one site for magnesium and one site for calcium.

Asfotase alfa can also be characterized as follows. From the N-terminus to the C terminus, asfotase alfa comprises: (1) the soluble catalytic domain of human tissue non-specific alkaline phosphatase (TNSALP) (UniProtKB/Swiss-Prot Accession No. P05186), (2) the human immunoglobulin G1 Fc domain (UniProtKB/Swiss-Prot Accession No. P01857) and (3) a deca-aspartate peptide (D₁₀) used as a bone-targeting domain (Nishioka et al. 2006 Mol Genet Metab 88:244-255). The protein associates into a homo-dimer from two primary protein sequences. This fusion protein contains 6 confirmed complex N-glycosylation sites. Five of these N-glycosylation sites are located on the sALP domain and one on the Fc domain. Another important post-translational modification present on asfotase alfa is the presence of disulfide bridges stabilizing the enzyme and the Fc-domain structure. A total of 4 intra-molecular disulfide bridges are present per monomer and 2 inter-molecular disulfide bridges are present in the dimer. One cysteine of the alkaline phosphatase domain is free.

Asfotase alfa may be used as an enzyme-replacement therapy for the treatment of hypophosphatasia (HPP). In patients with HPP, loss-of-function mutation(s) in the gene encoding TNSALP causes a deficiency in TNSALP enzymatic activity, which leads to elevated circulating levels of substrates, such as inorganic pyrophosphate (PPi) and pyridoxal-5′-phosphate (PLP). Administration of asfotase alfa to patients with HPP cleaves PPi, releasing inorganic phosphate for combination with calcium, thereby promoting hydroxyapatite crystal formation and bone mineralization, and restoring a normal skeletal phenotype. For more details on asfotase alfa and its uses in treatment, see PCT Publication Nos. WO 2005/103263 and WO 2008/138131

In some embodiments, the method provides an alkaline phosphatase (asfotase alfa) having improved enzymatic activity of the produced alkaline phosphatase (e.g., asfotase alfa) relative to an alkaline phosphatase produced by conventional means, by minimizing the concentration of metal ions having potential negative impact on activity or increasing the concentration of metal ions having potential positive impact on activity or both as described herein. Activity may be measured by any known method. Such methods include, e.g., those in vitro and in vivo assays measuring the enzymatic activity of the produced alkaline phosphatase (e.g., asfotase alfa) to substrates of an alkaline phosphatase, such as phosphoethanolamine (PEA), inorganic pyrophosphate (PPi) and pyridoxal 5′-phosphate (PLP).

In some embodiments, the alkaline phosphatase disclosed herein is encoded by a first polynucleotide which hybridizes under high stringency conditions to a second polynucleotide comparing the sequence completely complementary to a third polynucleotide encoding a polypeptide comprising the sequence as set forth in SEQ ID NO:1. Such high stringency conditions may comprise: pre-hybridization and hybridization in 6×SSC, 5× Denhardt's reagent, 0.5% SDS and 100 mg/ml of denatured fragmented salmon sperm DNA at 68° C.; and washes in 2×SSC and 0.5% SDS at room temperature for 10 minutes; in 2×SSC and 0.1% SDS at room temperature for 10 minutes; and in 0.1×SSC and 0.5% SDS at 65° C. three times for 5 minutes.

### Manufacturing Process

The alkaline phosphatase protein described herein (e.g., asfotase alfa) may be produced by mammalian or other cells, particularly CHO cells, using methods known in the art. Such cells may be grown in culture dishes, flask glasses, or bioreactors. Specific processes for cell culture and producing recombinant proteins are known in the art, such as described in Nelson and Geyer, 1991 Bioprocess Technol. 13:112-143 and Rea et al., Supplement to BioPharm International March 2008, 20-25. Exemplary bioreactors include batch, fed-batch, and continuous reactors. In some embodiments, the alkaline phosphatase protein is produced in a fed-batch bioreactor.

Cell culture processes have variability caused by, for example, variable physicochemical environment, including but not limited to, changes in pH, temperature, temperature changes, timing of temperature changes, cell culture media composition, cell culture nutrient supplements, raw material lot-to-lot variation, medium filtration material, bioreactor scale difference, gassing strategy (air, oxygen, and carbon dioxide), etc. As disclosed herein, the yield, relative activity profile, and glycosylation profile of manufactured alkaline phosphatase protein may be affected by alterations in one or more parameters.

For recombinant protein production in cell culture, the recombinant gene with the necessary transcriptional regulatory elements is first transferred to a host cell. Optionally, a second gene is transferred that confers to recipient cells a selective advantage. In the presence of the selection agent, which may be applied a few days after gene transfer, only those cells that express the selector gene survive. Two exemplary genes for selection are dihydrofolate reductase (DHFR), an enzyme involved in nucleotide metabolism, and glutamine synthetase (GS). In both cases, selection occurs in the absence of the appropriate metabolite (hypoxanthine and thymidine, in the case of DHFR, glutamine in the case of GS), preventing growth of nontransformed cells. In general, for efficient expression of the recombinant protein, it is not important whether the biopharmaceutical-encoding gene and selector genes are on the same plasmid or not.

Following selection, surviving cells may be transferred as single cells to a second cultivation vessel, and the cultures are expanded to produce clonal populations. Eventually, individual clones are evaluated for recombinant protein expression, with the highest producers being retained for further cultivation and analysis. From these candidates, one cell line with the appropriate growth and productivity characteristics is chosen for production of the recombinant protein. A cultivation process is then developed that is determined by the production needs and the requirements of the final product.

### Cells

Any mammalian cell or non-mammalian cell type, which can be cultured to produce a polypeptide, may be utilized in accordance with the present disclosure. Non-limiting examples of mammalian cells that may be used include, e.g., Chinese hamster ovary cells+/−DHFR (CHO, Urlaub and Chasin, 1980 Proc. Natl. Acad. Sci. USA, 77:4216); BALB/c mouse myeloma line (NSO/1, ECACC Accession No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., 1977 J. Gen Virol., 36:59); baby hamster kidney cells (BHK, ATCC CCL 10); mouse Sertoli cells (TM4, Mather, Biol. Reprod., 23:243-251 (1980)); monkey kidney cells (CV1 ATCC CCL 70); African green monkey kidney cells (VERO-76, ATCC CRL-I 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982, Annals N.Y. Acad. Sci. 383:44-68); MRC 5 cells; FS4 cells; and a human hepatoma line (Hep G2). In a particular embodiment, culturing and expression of polypeptides and proteins occurs from a Chinese Hamster Ovary (CHO) cell line.

Additionally, any number of commercially and non-commercially available hybridoma cell lines that express polypeptides or proteins may be utilized in accordance with the present disclosure. One skilled in the art will appreciate that hybridoma cell lines might have different nutrition requirements and/or might require different culture conditions for optimal growth and polypeptide or protein expression, and will be able to modify conditions as needed.

As noted above, in many instances the cells will be selected or engineered to produce high levels of protein or polypeptide. Often, cells are genetically engineered to produce high levels of protein, for example by introduction of a gene encoding the protein or polypeptide of interest and/or by introduction of control elements that regulate expression of the gene (whether endogenous or introduced) encoding the polypeptide of interest.

Seeding Density

In the present disclosure, Chinese Hamster Ovary(CHO) cells are inoculated, i.e., seeded, into the culture medium. Various seeding densities can be used. In some embodiments, a seeding density of 1.0×10⁴ cells/mL to 1.0×10⁷ cells/mL can be used. In some embodiments, a seeding density of 1.0×10⁵ cells/mL to 1.0×10⁶ cells/mL can be used. In some embodiments, a seeding density of 4.0×10⁵ cells/mL to 8.0×10⁵ cells/mL can be used. In some embodiments, a seeding density of 5.0×10⁵ cells/mL to 6.0×10⁵ cells/mL can be used. In some embodiments, a seeding density of 5.5×10⁵ cells/mL can be used. In some embodiments, increased seeding density can impact fragmentation of asfotase alfa quality, as measured by SEC. In some embodiments, the seeding density is controlled when inoculating in order to reduce the risk of fragment generation.

Temperature

Prior results indicated that temperature may have an impact on several parameters including growth rate, aggregation, fragmentation, and TSAC. In some embodiments, the temperature remains constant when culturing the CHO cells in the culture medium. In some embodiments, the temperature is about 30° C. to about 40° C., or about 35° C. to about 40° C. , or about 37° C. to about 39° C., or about 37.5° C. when culturing the CHO cells in the culture medium. In some embodiments, the temperature is constant for 40 to 200 hours after inoculation. In some embodiments, the temperature is constant for 50 to 150 hours, or 60 to 140 hours, or 70 to 130 hours, or 80 to 120 hours, or 90 to 110 hours after inoculation. In some embodiments, the temperature is constant for 80 to 120 hours after inoculation. In some embodiments, the temperature is constant for 90 hours, 92 hours, 94 hours, 96 hours, 98 hours, 100 hours, 102 hours, 104 hours, 106 hours, 108 hours or 110 hours after inoculation.

Temperature Shifting

Run times of cell culture processes, especially non-continuous processes (e.g., fed-batch processes in bioreactors), are usually limited by the remaining viability of the cells, which typically declines over the course of the run. Therefore, extending the length of time for cell viability is desired for improving recombination protein production. Product quality concerns also offer a motivation for minimizing decreases in viable cell density and maintaining high cell viability, as cell death can release sialidases to the culture supernatant, which may reduce the sialic acid content of the protein expressed. Protein purification concerns offer yet another motivation for minimizing decreases in viable cell density and maintaining high cell viability. Cell debris and the contents of dead cells in the culture can negatively impact one's ability to isolate and/or purify the protein product at the end of the culturing run. Thus, by keeping cells viable for a longer period of time in culture, there is a reduction in the contamination of the culture medium by cellular proteins and enzymes (e.g., cellular proteases and sialidases) that may cause degradation and ultimate reduction in the quality of the desired glycoprotein produced by the cells.

Many methods may be applied to achieve high cell viability in cell cultures. One involves lowering culture temperature following initial culturing at a normal temperature. For example, see Ressler et al., 1996, Enzyme and Microbial Technology 18:423-427). Generally, the mammalian or other types of cells capable of expressing a protein of interest are first grown under a normal temperature to increase cell numbers. Such “normal” temperatures for each cell type are generally around 37° C. (e.g., from about 35° C. to about 39° C., including, for example, 35.0° C., 35.5° C., 36.0° C., 36.5° C., 37.0° C., 37.5° C., 38.0° C., 38.5° C., and/or 39.0° C.). In one particular embodiment, the temperature for producing asfotase alfa is first set at about 37° C. When a reasonably high cell density is reached, the culturing temperature for the whole cell culture is then shifted (e.g., decreased) to promote protein production. In most cases lowering temperature shifts the cells towards the non-growth G1 portion of the cell cycle, which may increase cell density and viability, as compared to the previous higher-temperature environment. In addition, a lower temperature may also promote recombinant protein production by increasing the cellular protein production rate, facilitating protein post-translational modification (e.g., glycosylation), decreasing fragmentation or aggregation of newly-produced proteins, facilitating protein folding and formation of 3D structure (thus maintaining activity), and/or decreasing degradation of newly produced proteins. In some embodiments, the temperature is decreased 3° C., 4° C., 5° C., 6° C., 7° C., 8° C., 9° C., or 10° C. In some embodiments, the temperature is decreased to about 27° C., 28° C., 29° C., 30° C., 31° C., 32° C., 33° C., 34° C., or 35° C. In some embodiments, the lower temperature is from about 30° C. to about 35° C. (e.g., 30.0° C., 30.5° C., 31.0° C., 31.5° C., 32.0° C., 32.5° C., 33.0° C., 33.5° C., 34.0° C., 34.5° C., and/or 35.0° C.). In other embodiments, the temperature for producing asfotase alfa is first set to from about 35.0° C. to about 39.0° C. and then shifted to from about 30.0° C. to about 35.0° C. In one embodiment, the temperature for producing asfotase alfa is first set at about 37.0° C. and then shifted to about 30° C. In another embodiment, the temperature for producing asfotase alfa is first set at about 36.5° C. and then shifted to about 33° C. In yet another embodiment, the temperature for producing asfotase alfa is first set at about 37.0° C. and then shifted to about 33° C. In yet a further embodiment, the temperature for producing asfotase alfa is first set at about 36.5° C. and then shifted to about 30° C. In other embodiments, multiple (e.g., more than one) steps of temperature shifting may be applied. For example, the temperature may be lowered from 37° C. first to 33° C. and then further to 30° C.

The time for maintaining the culture at a particular temperature prior to shifting to a different temperature may be determined to achieve a sufficient (or desired) cell density while maintaining cell viability and an ability to produce the protein of interest. In some embodiments, the cell culture is grown under the first temperature until the viable cell density reaches about 10⁵ cells/mL to about 10⁷ cells/mL (e.g., 1×10⁵, 1.5×10⁵, 2.0×10⁵, 2.5×10⁵, 3.0×10⁵, 3.5×10⁵, 4.0×10⁵, 4.5×10⁵, 5.0×10⁵, 5.5×10⁵, 6.0×10⁵, 6.5×10⁵, 7.0×10⁵, 7.5×10⁵, 8.0×10⁵, 8.5×10⁵, 9.0×10⁵, 9.5×10⁵, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1×10⁷ cell/mL, or more) before shifting to a different temperature. In one embodiment, the cell culture is grown under the first temperature until the viable cell density reaches about 2.5 to about 3.4×10⁶ cells/mL before shifting to a different temperature. In another embodiment, the cell culture is grown under the first temperature until the viable cell density reaches about 2.5 to about 3.2×10⁶ cells/mL before shifting to a different temperature. In yet another embodiment, the cell culture is grown under the first temperature until the viable cell density reaches about 2.5 to about 2.8×10⁶ cells/mL before shifting to a different temperature.

In some embodiments, the cell culture is grown under 37° C. until the viable cell density reaches about 2.5-2.8×10⁶ cells/mL before shifting to 30° C. for protein production. In other embodiments, the cell culture is grown under 37° C. until the viable cell density reaches about 2.5-3.4×10⁶ cells/mL before shifting to 30° C. for protein production.

In some embodiments, the method of the present disclosure provides the temperature shift occurs 50 to 150 hours, or 60 to 140 hours, or 70 to 130 hours, or 80 to 120 hours, or 90 to 110 hours after inoculation. In some embodiments, the method of the present disclosure provides the temperature decreased about 80 hours to 150 hours after inoculation, about 90 hours to 100 hours after inoculation or about 96 hours after inoculation. In some embodiments, the temperature shift occurs 80 to 120 hours after inoculation. In some embodiments, the temperature shift occurs 90 hours, 92 hours, 94 hours, 96 hours, 98 hours, 100 hours, 102 hours, 104 hours, 106 hours, 108 hours or 110 hours after inoculation. In some embodiments, the temperature after the temperature shift is maintained until the CHO cells are harvested. In some embodiments, the CHO cells are harvested 10 days after inoculation. In some embodiments, the CHO cells are harvested 14 days after inoculation.

pH

Alteration of the pH of the growth medium in cell culture may affect cellular proteolytic activity, secretion, and protein production levels. Most of the cell lines grow well at about pH 7-8. Although optimum pH for cell growth varies relatively little among different cell strains, some normal fibroblast cell lines perform best at a pH 7.0-7.7 and transformed cells typically perform best at a pH of 7.0-7.4 (Eagle, 1973 The effect of environmental pH on the growth of normal and malignant cells. J Cell Physiol 82:1-8). In some embodiments, the pH of the culture medium for producing asfotase alfa is about pH 6.5-7.7 (e.g., 6.50, 6.55, 6.60, 6.65, 6.70, 6.75, 6.80, 6.85, 6.90, 6.95, 7.00, 7.05, 7.10, 7.15, 7.20, 7.25, 7.30, 7.35, 7.39, 7.40, 7.45, 7.50, 7.55, 7.60, 7.65, and 7.70). In some embodiments, the pH of the culture medium for producing asfotase alfa is about pH 7.20-7.60. In other embodiments, the pH of the culture medium for producing asfotase alfa is about pH 6.9-7.1. In one particular embodiment, the pH of the culture medium for producing asfotase alfa is about pH 6.9. In another embodiment, the pH of the culture medium for producing asfotase alfa is about pH 7.30. In yet another embodiment, the pH of the culture medium for producing asfotase alfa is about pH 7.39.

Culture Medium

In some embodiments, batch culture is used, wherein no additional culture medium is added after inoculation. In some embodiments, fed batch is used, wherein one or more boluses of culture medium are added after inoculation. In some embodiments, two, three, four, five or six boluses of culture medium are added after inoculation.

In various embodiments, alkaline phosphatase (e.g., asfotase alfa) is produced by a process in which extra boluses of culture medium are added to the production bioreactor. For example, one, two, three, four, five, six, or more boluses of culture medium may be added. In one particular embodiment, three boluses of culture medium are added. In various embodiments, such extra boluses of culture medium may be added in various amounts. For example, such boluses of culture medium may be added in an amount of about 20%, 25%, 30%, 33%, 40%, 45%, 50%, 60%, 67%, 70%, 75%, 80%, 90%, 100%, 110%, 120%, 125%, 130%, 133%, 140%, 150%, 160%, 167%, 170%, 175%, 180%, 190%, 200%, or more, of the original volume of culture medium in the production bioreactor. In one particular embodiment, such boluses of culture medium may be added in an amount of about 33%, 67%, 100%, or 133% of the original volume. In various embodiments, such addition of extra boluses may occur at various times during the cell growth or protein production period. For example, boluses may be added at day 1, day 2, day 3, day 4, day 5, day 6, day 7, day 8, day 9, day 10, day 11, day 12, or later in the process. In one particular embodiment, such boluses of culture medium may be added in every other day (e.g., at (1) day 3, day 5, and day 7; (2) day 4, day 6, and day 8; or (3) day 5, day 7, and day 9. In practice, the frequency, amount, time point, and other parameters of bolus supplements of culture medium may be combined freely according to the above limitation and determined by experimental practice.

Various culture mediums are available commercially. In some embodiments, the culture medium is selected from the group consisting of EX-CELL® 302 Serum-Free Medium; CD DG44 Medium; BD Select™ Medium; SFM4CHO Medium, or a combination thereof. In some embodiments, the culture medium comprises a combination of commercially available mediums, e.g., SFM4CHO Medium and BD Select™ Medium. In some embodiments, the culture medium comprises a combination of commercially available mediums, e.g., SFM4CHO Medium and BD Select™ Medium, at a ratio selected from 90/10, 80/20, 75/25, 70/30, 60/40, or 50/50. In some embodiments, the culture medium comprises a combination of of commercially available mediums, e.g., SFM4CHO Medium and BD Select™ Medium, at a ratio 75/25.

### Nutrient Supplement

Various nutrient supplements, also referred to as “feed media,” are commercially available and are known to those of skill in the art. Nutrient supplements includes a media (distinct from the culture media) added to a cell culture after inoculation has occurred. In some instances, the nutrient supplement can be used to replace nutrients consumed by the growing cells in the culture. In some embodiments, the nutrient supplement is added to optimize production of a desired protein, or to optimize activity of a desired protein. Numerous nutrient supplements have been developed and are available commercially. While the expressed purpose of the nutrient supplements are to increase an aspect of process development, no universal nutrient supplement exists that works for all cells and/or all proteins produced. The selection of a scalable and appropriate cell culture nutrient supplement that can work in combination with the desired cell line, protein produced and a given base medium to achieve the desired titer and growth characteristics is not routine. The typical approach of screening multiple commercially available nutrient supplements and identifying the most appropriate supplement with a specific cell line, specific protein produced and base medium combination may not be successful due to the myriad of variables present in the cell culture process. In some embodiments, the nutrient supplement is selected from the group consisting of Efficient Feed C+ AGT™ Supplement (Thermo Gisher Scientific, Waltham, Mass.), a combination of Cell Boost™ 2+Cell Boost™ 4 (GE Healthcare, Sweden), a combination of Cell Boost™ 2+Cell Boost™ 5 (GE Healthcare, Sweden), Cell Boost™ 6 (GE Healthcare, Sweden), and Cell Boost™ 7a+Cell Boost™ 7b (GE Healthcare, Sweden), or combinations thereof.

By way of example of the variety of components present in nutrient supplements, reference is made to Table A, which lists the components of the commercially available supplements Cell Boost™ produced by GE Healthcare.

TABLE A

| | | | | Trace | Growth | Hypoxanthine/ | | Synthetic | |
|---|---|---|---|---|---|---|---|---|---|
| | Amino Acids | Vitamins | Glucose | Elements | Factors | Thymidine | Lipids | Cholesterol | L-glutamine |
| Cell Boost 1 | X | X | X | | | | | | No |
| Cell Boost 2 | X | | X | | | | | | No |
| Cell Boost 3 | X | X | X | X | | X | | | No |
| Cell Boost 4 | X | X | X | X | X | | X | X | No |
| Cell Boost 5 | X | X | X | X | X | X | X | X | No |
| Cell Boost 6 | X | X | X | X | X | X | X | X | No |
| Cell Boost 7a | X | X | X | X | No | No | No | No | No |
| Cell Boost 7b | X | | No | | No | No | No | No | No |

Cell Boost™ 7a can be described as a first animal-derived component-free (ADCF) nutrient supplement comprising one or more amino acids, vitamins, salts, trace elements, poloxamer and glucose, wherein the first ADCF nutrient supplement does not comprise hypoxanthine, thymidine, insulin, L-glutamine, growth factors, peptides, proteins, hydrolysates, phenol red and 2-mercaptoethanol. Cell Boost™ 7a is a chemically defined supplement. The phrase “animal-derived component-free” or “ADCF” refers to a supplement in which no ingredients are derived directly from an animal source, e.g., are not derived from a bovine source. In some embodiments, the nutrient supplement is Cell Boost™ 7a.

Cell Boost™ 7b can be described as a second ADCF nutrient supplement comprising one or more amino acids, wherein the second ADCF nutrient supplement lacks hypoxanthine, thymidine, insulin, L-glutamine, growth factors, peptides, proteins, hydrolysates, phenol red, 2-mercaptoethanol and poloxamer. Cell Boost™ 7b is a chemically defined supplement. In some embodiments, the nutrient supplement is Cell Boost™ 7b.

In some embodiments, combinations of commercially available nutrient supplements are used. The term “nutrient supplement” refers to both a single nutrient supplement, as well as combinations of nutrient supplements. For example, in some embodiments a combination of nutrient supplements includes a combination of Cell Boost™ 7a and Cell Boost™ 7b.

In various embodiments, alkaline phosphatase (e.g., asfotase alfa) is produced by a process in which extra additions of nutrient supplement are added to the production bioreactor. In some embodiments, the nutrient supplement is added over a period of time, e.g., over a period of time ranging from 1 minute to 2 hours. In some embodiments, the nutrient supplement is added in a bolus. For example, one, two, three, four, five, six, or more boluses of nutrient supplement may be added. In one particular embodiment, one, two or three boluses of nutrient supplement are added. In some embodiments, the nutrient supplement is added a more than 2 different times, e.g., 2 to 6 different times. In some embodiments, the nutrient supplement is added at 4 different times. In various embodiments, such extra boluses of nutrient supplement may be added in various amounts. For example, such boluses of nutrient supplement may be added in an amount of about 1% to 20%, 1% to 10% or 1% to 5% (w/v) of the original volume of culture medium in the production bioreactor. In one particular embodiment, such boluses of nutrient supplement may be added in an amount of 1% to 20%, 1% to 10% or 1% to 5% (w/v) of the original volume.

In some embodiments, a combination of nutrient supplements is used, and the first nutrient supplement, e.g., Cell Boost™ 7a, is added at a concentration of 0.5% to 4% (w/v) of the culture medium. In some embodiments, a combination of nutrient supplements is used, and the first nutrient supplement, e.g., Cell Boost™ 7a, is added at a concentration of 2% (w/v) of the culture medium. In some embodiments, a combination of nutrient supplements is used, and the second nutrient supplement, e.g., Cell Boost™ 7b, is added at a concentration of 0.05% to 0.8% (w/v) of the culture medium. In some embodiments, a combination of nutrient supplements is used, and the first nutrient supplement, e.g., Cell Boost™ 7b, is added at a concentration of 0.2% (w/v) of the culture medium. In specific embodiments wherein a combination of nutrient supplements include Cell Boost™ 7a and Cell Boost™ 7b, a boluses of nutrient supplement may be added in an amount of 1% to 20%, 1% to 10% or 1% to 5% (w/v) of the original volume. In specific embodiments wherein a combination of nutrient supplements includes Cell Boost™ 7a and Cell Boost™ 7b, a bolus of Cell Boost™ 7a nutrient supplement may be added in an amount of 1% to 20%, 1% to 10% or 1% to 5% (w/v) of the original volume, and a boluses of Cell Boost™ 7b nutrient supplement may be added in an amount of 0.1% to 2%, 0.1% to 1% or 0.1% to 0.5% (w/v) of the original volume.

In some embodiments where multiple additions, e.g., boluses, of nutrient supplements are added to the cell culture, the total addition of the first nutrient supplement, e.g., Cell Boost™ 7a, is added at a concentration of 5% to 20% (w/v) of the culture medium. In some embodiments where multiple additions, e.g., boluses, of nutrient supplements are added to the cell culture, the total addition of the first nutrient supplement, e.g., Cell Boost™ 7a, is added at a concentration of 12% (w/v) of the culture medium. In some embodiments where multiple additions, e.g., boluses, of nutrient supplements are added to the cell culture, the total addition of the second nutrient supplement, e.g., Cell Boost™ 7b, is added at a concentration of 0.5% to 2% (w/v) of the culture medium. In some embodiments where multiple additions, e.g., boluses, of nutrient supplements are added to the cell culture, the total addition of the second nutrient supplement, e.g., Cell Boost™ 7b, is added at a concentration of 1.2% (w/v) of the culture medium.

In various embodiments, such addition of extra boluses may occur at various times after inoculation. For example, boluses may be added at day 1, day 2, day 3, day 4, day 5, day 6, day 7, day 8, day 9, day 10, day 11, day 12, or later after inoculation. In some embodiments, the nutrient supplement is added 1 to 3 days after inoculation and 3 to 5 days after inoculation. In some embodiments, the nutrient supplement is added 1 to 3 days after inoculation, 3 to 5 days after inoculation, 5 to 7 days after inoculation and 7 to 9 days after inoculation. In some embodiments, the nutrient supplement is added 2 days after inoculation, 4 days after inoculation, 6 days after inoculation and 8 days after inoculation. In one particular embodiment, such boluses of nutrient supplement may be added in every other day (e.g., at (1) day 3, day 5, and day 7; (2) day 4, day 6, and day 8; or (3) day 5, day 7, and day 9. In one embodiments, the nutrient supplement is added on days 2, 4, 6, 8 and 10 after inoculation. In specific embodiments wherein a combination of nutrient supplements includes Cell Boost™ 7a and Cell Boost™ 7b, boluses of nutrient supplement can be added on days 2, 4, 6, 8 and 10. In practice, the frequency, amount, time point, and other parameters of bolus supplements of nutrient supplement may be combined freely according to the above limitation and determined by experimental practice.

### Zinc Supplementation

Zinc ions are known to be important for alkaline phosphatase (e.g., asfotase alfa) stability as it helps to maintain their structure and activity. For example, two zinc atoms associate with one placental alkaline phosphatase molecule (Helene Le Du et al. 2001 J. Biol. Chem. 276:9158-9165). Based on this ratio, for the titer of lg/L asfotase alfa produced by the exemplary manufacturing process developed in small-scale models, approximately 20 μM zinc is needed for asfotase alfa activity. Although 20 μM zinc is theoretically sufficient for producing functional asfotase alfa (i.e., 2 zinc ions per active enzyme), the actual zinc supplementation may require significantly higher zinc concentrations (e.g., 150 μM) in alkaline phosphatase (e.g., asfotase alfa, TNALP, PALP, GCALP, IAP, or fusion/variant proteins thereof) manufacturing processes.

In some embodiments, the method disclosed herein further comprises adding zinc into said culture medium during production of the recombinant polypeptide. In some embodiments, zinc may be added to provide a zinc concentration of from about 1 to about 300 μM in said culture medium. In one embodiment, zinc may be added to provide a zinc concentration of from about 10 to about 200 μM (e.g., 10, 20, 30, 40, 50, 60, 70, 80, 90, 100, 110, 120, 130, 140, or 150 μM) in the culture medium. In some embodiments, zinc is added to provide a zinc concentration in the culture medium of from about 25 μM to about 150 μM, or about 60 μM to about 150 μM. In one embodiment, zinc is added to provide a zinc concentration in the culture medium of from about 30, 60, or 90 μM of zinc. In one embodiment, zinc is added to provide a zinc concentration in the culture medium of 80 μM, 90 μM, or 100 μM of zinc, preferably about 90 μM zinc. In some embodiments, the zinc is added into said culture medium in a bolus, continuously, semi-continuously, or combinations thereof. In some embodiments, zinc is added one day, two days, three days, four days, five days, six days, seven days, eight days, nine days, ten days, eleven days, twelve days, and/or thirteen days after inoculation. In some embodiments, one, two, three, four, five, six, seven, eight, nine, ten, eleven, twelve, or thirteen boluses of zinc are added after inoculation. In some embodiments, zinc is added in the form of zinc sulfate. Zinc concentration is determined by Zn²⁺ concentration, and may be provided with any physiologically appropriate counter-ion as a salt.

### Harvest

Prior studies suggested that delaying harvest timing was associated with a viability and TSAC decline, so harvest timing can have a potential impact on other CQAs. In various embodiments, alkaline phosphatase (e.g., asfotase alfa) is harvested at a time point of about 200 hr, 210 hr, 220 hr, 230 hr, 240 hr, 250 hr, 260 hr, 264 hr, 270 hr, 280 hr, 288 hr (i.e., 12 days), or more than 12 days. In particular embodiments, alkaline phosphatase (e.g., asfotase alfa) is harvested at a time point of about 10 days or about 14 days.

### Downstream Processes

The term “downstream process(es)” used herein is generally referred to the whole or part(s) of the processes for recovery and purification of the alkaline phosphatases (e.g., asfotase alfa) produced from sources such as culture cells or fermentation broth, including the recycling of salvageable components and the proper treatment and disposal of waste.

Generally, downstream processing brings a product from its natural state as a component of a tissue, cell or fermentation broth through progressive improvements in purity and concentration. For example, the removal of insolubles may be the first step, which involves the capture of the product as a solute in a particulate-free liquid (e.g., separating cells, cell debris or other particulate matter from fermentation broth). Exemplary operations to achieve this include, e.g., filtration, centrifugation, sedimentation, precipitation, flocculation, electro-precipitation, gravity settling, etc. Additional operations may include, e.g., grinding, homogenization, or leaching, for recovering products from solid sources, such as plant and animal tissues. The second step may be a “product-isolation” step, which removes components whose properties vary markedly from that of the desired product. For most products, water is the chief impurity and isolation steps are designed to remove most of it, reducing the volume of material to be handled and concentrating the product. Solvent extraction, adsorption, ultrafiltration, and precipitation may be used alone or in combinations for this step. The next step is about product purification, which separates contaminants that resemble the product very closely in physical and chemical properties. Possible purification methods include, e.g., affinity, ion-exchange chromatography, hydrophobic interaction chromatography, mixed-mode chromatography, size exclusion, reversed phase chromatography, ultrafiltration-diafiltration, crystallization and fractional precipitation. In some embodiments, the chromatography step comprises at least one of harvest clarification, ultrafiltration, diafiltration, viral inactivation, affinity capture, and combinations thereof.

In some embodiments, the methods described herein further comprise measuring recombinant alkaline phosphatase activity. In some embodiments, the activity is selected from a method selected from at least one of a pNPP-based alkaline phosphatase enzymatic assay and an inorganic pyrophosphate (PPi) hydrolysis assay. In some embodiments, at least one of the recombinant alkaline phosphatase K_{cat} and Kₘ values increases in an inorganic pyrophosphate (PPi) hydrolysis assay. In some embodiments, the method comprises determining an integral of viable cell concentration (IVCC). In some embodiments, the IVCC is increased by from about 3.0-fold to about 6.5-fold compared to the method in the absence of steps (iii) and (iv) as described herein.

The last step may be used for product polishing, the processes which culminate with packaging of the product in a form that is stable, easily transportable and convenient. Storage at 2-8° C., freezing at −20° C. to −80° C., crystallization, desiccation, lyophilization, freeze-drying and spray drying are exemplary methods in this final step. Depending on the product and its intended use, product polishing may also sterilize the product and remove or deactivate trace contaminants (e.g., viruses, endotoxins, metabolic waste products, and pyrogens), which may compromise product safety.

Product recovery methods may combine two or more steps discussed herein. For example, expanded bed adsorption (EBA) accomplishes removal of insolubles and product isolation in a single step. For a review of EBA, see Kennedy, Curr Protoc Protein Sci. 2005 June; Chapter 8: Unit 8.8. In addition, affinity chromatography often isolates and purifies in a single step.

For a review of downstream processes for purifying a recombinant protein produced in culture cells, see Rea, 2008 Solutions for Purification of Fc-fusion Proteins. Bio Pharm Int. Supplements March 2:20-25. The downstream processes for alkaline phosphatases disclosed herein may include at least one, or any combination, of the following exemplary steps:

a harvest clarification process. In this step, the intact cells and cell debris are removed by sterile filtration and the product (i.e., the produced alkaline phosphatase) is recovered. Possible used solutions in this step may include a recovery buffer (e.g., 50 mM Sodium Phosphate, 100 mM NaCl, pH 7.50);

a post-harvest ultrafiltration (UF) and/or diafiltration (DF) process. The purpose for this step is for concentration and buffer dilution. Exemplary steps for the UF process include, e.g., pre-use cleaning/storage of the filter membrane, post-clean/post-storage flush, equilibration (e.g., with a buffer containing 50 mM Sodium Phosphate, 100 mM NaCl, pH 7.50), loading, concentration, dilution/flush/recovery (e.g., with a buffer containing 50 mM Sodium Phosphate, 100 mM NaCl, pH 7.50), and post-use flush/clean/storage of the filter membrane;

a solvent/detergent viral inactivation process to chemically inactivate viral particles. Exemplary solvent/detergent may contain 10% Polysorbate 80, 3% TNBP, 50 mM Sodium Phosphate, and 100 mM NaCl;

a certain type of column chromatography to further purify the product and/or separate the impurities/contaminants, such as gel filtration chromatography, ion exchange chromatography, reversed-phase chromatography (RP), affinity chromatography, expanded bed adsorption (EBA), mixed-mode chromatography and hydrophobic interaction chromatography (HIC). Affinity capture process, e.g., Protein A chromatography, may be used to capture the product (i.e., the alkaline phosphatase, such as asfotase alfa). For example, a process of GE Healthcare Mab Select SuRe Protein A chromatography may be used. HIC chromatography may use Butyl Sepharose or CAPTO® Butyl agarose columns. Exemplary buffers and solutions used in Protein A chromatography include, e.g., equilibration/wash buffer (e.g., 50 mM Sodium Phosphate, 100 mM NaCl, pH 7.50), elution buffer (e.g., 50 mM Tris, pH 11.0), strip buffer (e.g., 100 mM Sodium Citrate, 300 mM NaCl, pH 3.2), flushing buffer, cleaning solution (e.g., 0.1 M NaOH), etc. Exemplary buffers and solutions used in a CAPTO® Butyl agarose HIC process include, e.g., loading dilution buffer/pre-equilibration buffer (e.g., 50 mM sodium phosphate, 1.4 M sodium sulfate, pH 7.50), equilibration buffer/wash buffer/elution buffer (e.g., all containing sodium phosphate and sodium sulfate), strip buffer (e.g., containing sodium phosphate), etc. Exemplary buffers and solutions used in a Butyl HIC process include, e.g., loading dilution buffer/pre-equilibration buffer (e.g., 10 mM HEPES, 2.0 M ammonium sulfate, pH 7.50), equilibration buffer/wash buffer(s)/elution buffer (e.g., all containing sodium phosphate or HEPES and ammonium sulfate), and strip buffer (e.g., containing sodium phosphate);

a post-HIC UF/DF process for, e.g., product concentration and/or buffer exchange. Exemplary buffers and solutions used in this process include, e.g., equilibration buffer (e.g., 20 mM Sodium Phosphate, 100 mM NaCl, pH 6.75), diafiltration buffer (20 mM Sodium Phosphate, 100 mM NaCl, pH 6.75), etc.;

a viral reduction filtration process to further remove any viral particles;

a mixed-mode chromatography, such as CAPTO® Adhere agarose chromatography. Commercially available mixed-mode materials include, e.g., resins containing hydrocarbyl amine ligands (e.g., PPA Hypercel and HEA Hypercel from Pall Corporation, Port Washington, N.Y.), which allow binding at neutral or slightly basic pH, by a combination of hydrophobic and electrostatic forces, and elution by electrostatic charge repulsion at low pH (see Brenac et al., 2008 J Chromatogr A. 1177:226-233); resins containing 4-mercapto-ethyl-pyridine ligand (MEP Hypercel, Pall Corporation), which achieves hydrophobic interaction by an aromatic residue and the sulphur atom facilitates binding of the target protein by thiophilic interaction (Lees et al., 2009 Bioprocess Int. 7:42-48); resins such as CAPTO® MMC mixed-mode chromatography and CAPTO® adhere agarose chromatography (GE Healthcare, Amersham, UK) containing ligands with hydrogen bonding groups and aromatic residues in the proximity of ionic groups, which leads to the salt-tolerant adsorption of proteins at different conductivities (Chen et al., 2010 J Chromatogr A. 1217:216-224); and other known chromatography materials, such as affinity resins with dye ligands, hydroxyapatite, and some ion-exchange resins (including, but not limited to, Amberlite CG 50 (Rohm & Haas, Philadelphia, Pa.) or Lewatit CNP 105 (Lanxess, Cologne, Del.). For an exemplary agarose HIC chromatography step, exemplary buffers and solutions used in this process include, e.g., pre-equilibration buffer (e.g., 0.5 M Sodium Phosphate, pH 6.00), equilibration/wash buffer (e.g., 20 mM Sodium Phosphate, 440 mM NaCl, pH 6.50), load titration buffer (e.g., 20 mM Sodium Phosphate, 3.2 M NaCl, pH 5.75), pool dilution buffer (e.g., 25 mM Sodium Phosphate, 150 mM NaCl, pH 7.40), and strip buffer (0.1 M Sodium Citrate, pH 3.20;

a virus filtration for viral clearance (by, e.g., size exclusion). Exemplary buffers and solutions used in this process include, e.g., pre-use and post-product flush buffer (e.g., 20 mM Sodium Phosphate, 100 mM NaCl, pH 6.75);

a formulation (may comprise UF/DF process for, e.g., concentration and/or buffer exchange) process. Exemplary buffers and solutions used in this process include, e.g., filter flush/equilibration/diafiltration/recovery buffer (e.g., 25 mM Sodium Phosphate, 150 mM NaCl, pH 7.40); and

a bulk fill process comprising sterile filtration (exemplary filters are Millipak 60 or Equivalent sized PVDF filters (EMD Millipore, Billerica, Mass.).

In some embodiments, the steps used for producing, purifying, and/or separating the alkaline phosphatase from the culture cells, as disclosed herein, further comprise at least one of steps selected from the group consisting of: a harvest clarification process (or a similar process to remove the intact cells and cell debris from the cell culture), an ultrafiltration (UF) process (or a similar process to concentrate the produced alkaline phosphatase), a diafiltration (DF) process (or a similar process to change or dilute the buffer comprising the produced alkaline phosphatase from previous processes), a viral inactivation process (or a similar process to inactivate or remove viral particles), an affinity capture process (or any one of chromatography methods to capture the produced alkaline phosphatase and separate it from the rest of the buffer/solution components), a formulation process and a bulk fill process. In one embodiment, the steps for producing, purifying, and/or separating the alkaline phosphatase from the culture cells, as disclosed herein, comprise at least a harvest clarification process (or a similar process to remove the intact cells and cell debris from the cell culture), a post-harvest ultrafiltration (UF) process (or a similar process to concentrate the produced alkaline phosphatase), a post-harvest diafiltration (DF) process (or a similar process to change or dilute the buffer comprising the produced alkaline phosphatase from previous processes), a solvent/detergent viral inactivation process (or a similar process to chemically inactivate viral particlesan intermediate purification process (such as hydrophobic interaction chromatography (HIC) or any one of chromatography methods to capture the produced alkaline phosphatase and separate it from the rest of the buffer/solution components), a post-HIC UF/DF process (or a similar process to concentrate and/or buffer exchange for the produced alkaline phosphatase), a viral reduction filtration process (or a similar process to further remove any viral particles or other impurities or contaminants); a mixed-mode chromatography (such as CAPTO® Adhere agarose chromatography, or a similar process to further purify and/or concentrate the produced alkaline phosphatase), a formulation process and a bulk fill process. In one embodiment, the separating step of the method provided herein further comprises at least one of harvest clarification, ultrafiltration, diafiltration, viral inactivation, affinity capture, HIC chromatography, mixed-mode chromatography and combinations thereof.

All references cited herein are incorporated by reference in their entirety. Although the foregoing disclosure has been described in some detail by way of illustration and example for purposes of clarity of understanding, it is apparent to those skilled in the art that certain minor changes and modifications will be practiced. Therefore, the description and examples should not be construed as limiting the scope of the disclosure.

### EXAMPLES

### Example 1: Evaluation of Culture Media, Nutrient Supplements, and Zinc Supplementation on Asfotase Alfa Productivity

A standard grown process “Control Process,” was developed by increasing the CHO feed amount used in previous processes from 0.5% (w/v) to 2.0% (w/v). Control Process had an average active titer of 0.12 g/L. Control Process had a considerably lower integral of viable cell concentration (IVCC) and volumetric titer compared with other commercial CHO-based upstream processes. Earlier efforts including supplementing complex nutrients and increasing existing feed amount had little or no success. Additionally, a temperature shift in Control Process was important to achieve target sialylation and to maximize activity levels.

Therefore, further process improvements were investigated with a target of at least two-fold process yield increase.

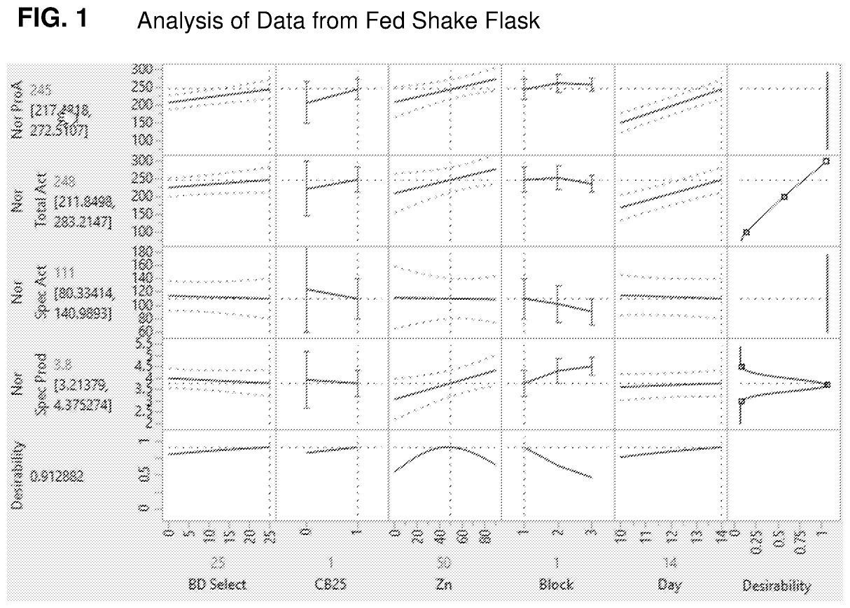

Preliminary studies evaluated the effect of alternative culture media, complex feed supplements, and metal supplementation in increasing the overall integral of viable cell concentration (IVCC), productivity, and activity levels (FIG. 1). In a preliminary study, the nutrient supplement (Cell Boost 2+Cell Boost 5) led to enhanced cell growth, sustained cell longevity, and two-fold increase of protein A bindable titer on day 14 in comparison to harvest day 10 in Control Process. However, the specific activity was 20% lower in the Cell Boost 2/5 test condition than in Control Process. As a result, the final volumetric activity was increased by only 60% on day 14 in the Cell Boost 2/5 test condition in comparison to Control Process.

The incorporation of zinc in asfotase alfa has been evaluated in relation to the specific activity of asfotase alfa. Upon investigation, the molar ratio of zinc to asfotase alpha used in the studying employing nutrient supplement (Cell Boost 2+Cell Boost 5) (molar ratio of 9) was closer to that of previous, historical processes (molar ratio of 8) than to Control Process (molar ratio of 19) (data not shown). The increased molar ratio of 19 for Control Process led to the investigation of zinc supplementation with Cell Boost nutrient additives.

A follow-up shake flask study evaluated the impact of zinc sulfate supplementation on enhancing the specific activity in the process. Among the conditions tested, the total activity was highest under the 90 μM zinc sulfate condition (˜97% higher than the Control Process condition). The process providing the second highest total activity was the 30 μM zinc sulfate condition (˜74% higher than the Control Process condition). In this study, all zinc sulfate was supplemented on Day 0. While the 30 μM zinc sulfate condition showed cell growth comparable to the Control Process through Day 4 and superior to the Control Process from Day 6 to Day 14, the 90 μM zinc sulfate condition showed lower VCD than the Control Process through Day 6 and comparable VCD Day 8 through Day 14, indicating the amount and the addition timing for zinc sulfate supplementation play a role in cell growth and viability.

Another study evaluating culture media effects demonstrated that when BD Select CHO Medium was included in the culture medium at concentrations of 25% and 50% (v/v), a higher peak viable cell density was achieved than the control condition (100% SFM4CHO medium) with comparable cell viability up to at least day 7 (which was the longest duration tested in this study). The average titer measurement for the 25% BD Select condition was 44% higher than Control Process, and the average specific productivity under the 25% BD Select condition was 30% higher than Control Process.

A subsequent study evaluated the impact of zinc sulfate (the amount and addition timing) and BD Select supplementation on productivity in 2 L bioreactors. The data from this study, as well as the data from the culture media evaluation study (Example 2) and the Zn supplementation study (Example 1), were analyzed to maximize total asfotase alfa activity and to target specific productivity that is comparable to the Control Process (FIG. 1). All four parameters (BD Select, Cell Boost 2+5, Zinc, and harvest day) had statistically significant effects on either total activity or specific productivity (FIG. 1). Cell Boost 2+5 had statistically significant effects on both total activity and on specific productivity (FIG. 1).

Readouts from these preliminary studies led to a study design evaluating the potential impact of supplementing nutrient feeds and delaying temperature shift on subsequent volumetric productivity.

### Example 2: Evaluation of Various Culture Media for Manufacturing Processes for Asfotase Alfa

As described herein, an improved manufacturing process to produce alkaline phosphatases (e.g., asfotase alfa (sTNALP-Fc-D₁₀)) was developed. Stable CHO cell lines expressing asfotase alfa were developed using a gene expression system, e.g., the GS or the DHFR gene expression system. Secondary clones were derived from high producing primary clones in a single round of limited dilution cloning and a final cell line was selected.

Three commercially available culture media and a mixture of two culture media were evaluated for cells expressing asfotase alfa. An exemplary manufacturing process is described below. A vial of the Master Cell Bank was thawed and the entire volume of the vial was re-suspended and grown in a culture medium of either EX-CELL® 302 Serum-Free Medium (Signam Aldrich, St. Louis, Mo.), CD DG44 Medium (ThermoFisher Scientific, Waltham, Mass.), BD Select Medium (BD Biosciences, San Jose, Calif.), or a mixture of BD Select Medium with SFM4CHO Medium (Hyclone, Logan Utah) as show in Table 1.

TABLE 1
Shake Flask Operating Conditions

| | | | Tempera- | | |
|---|---|---|---|---|---|
| | | Working | ture Set | CO₂ | |
| Media | | Vol | Point | Set | Passage |
| tested | Vessel | (mL) | (° C.) | Point % | Day |
| SFM4CHO | 1 L SF or | 100 mL | 36.5 | 5 | 3-4 |
| CD DG44 | 0.5 L SF | | (±1) | | |
| EXCELL ® 302 | | | | | |
| BD Select | | | | | |

Cells using EX-CELL® 302 Serum-Free Medium and CD DG44 Medium as their culture medium did not adapt well in seed train evaluation. Therefore, these media were not further tested. Cells grown using BD Select Medium appropriately adapted in seed train evaluation. During the seed train evaluation transition, cells grown using a mixture of BD Select Medium (25%) with SFM4CHO Medium (75%) achieved the best results (data not shown). Therefore, BD Select/SFM4CHO medium was used as the culture medium in further production bioreactor studies.

### Example 3: Evaluation of Nutrient Supplements on Cell Growth and Protein Production

Various chemically defined, commercially available nutrient feed supplements were evaluated against the Control Process (Table 3) to maximize yield without disrupting the integrity of the final alkaline phosphatase product. The cell culture process parameters for bioreactor control for this study are presented in Table 2.

Efficient Feed C+ AGT™ Supplement (Thermo Gisher Scientific, Waltham, Mass.), a combination of Cell Boost™ 2+Cell Boost™ 4 (GE Healthcare, Sweden), a combination of Cell Boost™ 2+Cell Boost™ 5 (GE Healthcare, Sweden), Cell Boost™ 6 (GE Healthcare, Sweden), and Cell Boost™ 7a+Cell Boost™ 7b (GE Healthcare, Sweden) were all investigated as outlined in Table 3. The nutrient supplements were evaluated in either in 2 L Sartorius production bioreactors or 1 L shake flasks, with and without a temperature shift.

TABLE 2
Cell Culture Process Parameters in 2 L Bioreactors

| Process Parameters | Setpoint or control strategy |
|---|---|
| Working volume | 2 L |
| post inoculation | |
| Seeding density | 5.5 × 10⁵ cells/mL |
| Culture Medium | BD Select Medium (25% w/v) and SFM4CHO |
| | Medium (76% w/v) |
| Agitation rate | 304 rpm (to match P/V 60 W/m³) |
| pH setpoint | 6.90 without dead band (P- 30%; I-1000s; D-0) |
| DO setpoint | 40% |
| Temperature setpoint | 37.0° C. or 30° C. post temperature shift |
| | (temperatures shift follows profile) |
| Gassing strategy | Air flow 6 mL/min + oxygen as needed up to 100 |
| | mL/min |
| 200 mM glutamine | If glutamine is <1.2 mM, add 20 mL of glutamine |
| addition | stock (200 mL) daily until the temperature is |
| | shifted. |
| 150 mM ZnSO₄ | 0.4 mL per bolus on day 4, 7 and 10 |
| addition | |
| CHO feed addition | 0.5% v/v (or 10.4 g) per bolus on days 2, 4, 6, |
| | and 8 in all bioreactors. |
| Additional Feed | See Table 3 |
| Supplmentation | |
| Antifoam | 1 mL/bolus only as needed. |
| 400 g/L glucose | If glucose level is below 2 g/L, add 10 mL |
| addition | glucose solution. |
| Partial Harvest | 2 × 100 mL on day 10 and on day 14 from all |
| | tanks |

TABLE 3
Study Design for Potential Process Improvements
in Nutrient Supplements

| | | | | | Temp |
|---|---|---|---|---|---|
| Bio- | | | | | Shift |
| reac- | | | | | Tim- |
| tor | Production | CHO | Nutrient | 150 mM | ing |
| ID | Medium | Feed | Supplements | ZnSO₄ | (h) |
| 1 | SFM4CHO | 0.5% (v/v) | N/A | N/A | 60 |
| 2 | medium + | or 10.4 g | | | |
| | Gln | on day 2, | | | |
| 3 | SFM4CHO | 4, 6 and | Cell Boost 2 + 5 | 0.4 mL on | 96 |
| | | 8 | (CB25) | day 4, 7, | |
| 4 | medium | | 3%(w/v) or 60 g on | & 10 | |
| | (75%) + | | day 2, 6, & 10 | | |
| 5 | BDSelect | | Cell Boost 6 (CB6) | | 96 |
| 6 | (25%) + | | 3%(w/v) or 60 g on | | N/A |
| | Gln | | day 2, 6, & 10 | | |
| 7 | | | Cell Boost 7a | | 96 |
| | | | (CB7a) | | |
| 8 | | | 2%(w/v) or 40 g on | | N/A |
| | | | day 2, 4, 6, 8, & | | |
| | | | 10 Cell Boost 7b | | |
| | | | (CB7b) 0.2%(w/v) | | |
| | | | or 4 mL on day 2, | | |
| | | | 4, 6, 8, & 10 | | |
| 9 | SFM4CHO | 2% (v/v) | Cell Boost 2 + 4 | N/A | 96 |
| | medium + | or 4 mL | (CB24) | | |
| | Gln | on day 2 | 3%(v/v) or 6 mL | | |
| | | | on days 2, 6, & | | |
| | | | 10 | | |
| 10 | SFM4CHO | 2% (v/v) | Efficient Feed C + | N/A | 96 |
| | medium + | or 4 mL | AGT ™3%(v/v) or | | |
| | Gln | on day 2 | 6 mL on days 2, 6, | | |
| | | | & 10 | | |

Experiments in Bioreactor ID Nos. 1-8 were performed 2 L bioreactors, and experiments in Bioreactor ID Nos 9 and 10 were performed in 1 L shake flasks. Cell culture retains (day 8, day 10, day 12 and day 14) were centrifuged and ProA bindable titer was determined. Partial harvests of 200 mL were performed for each bioreactor on day 10 and day 14. Samples were centrifuged (3000 g, 10 min), 0.2 μm filtered, stored at −80° C., and high throughput ProA purified. Overall specific productivity was calculated using ProA titer at harvest (day 10 and day 14) divided by IVCC on that day. Protein activity levels were measured using an enzyme based assay. Process impurity and sialylation levels were measured in the ProA purified harvest samples. Clarified cell culture broth was analyzed for pNPP activity, SEC-HPLC analysis was performed on ProA purified samples, and the ProA purified, buffer-exchanged samples were analyzed for TSAC.

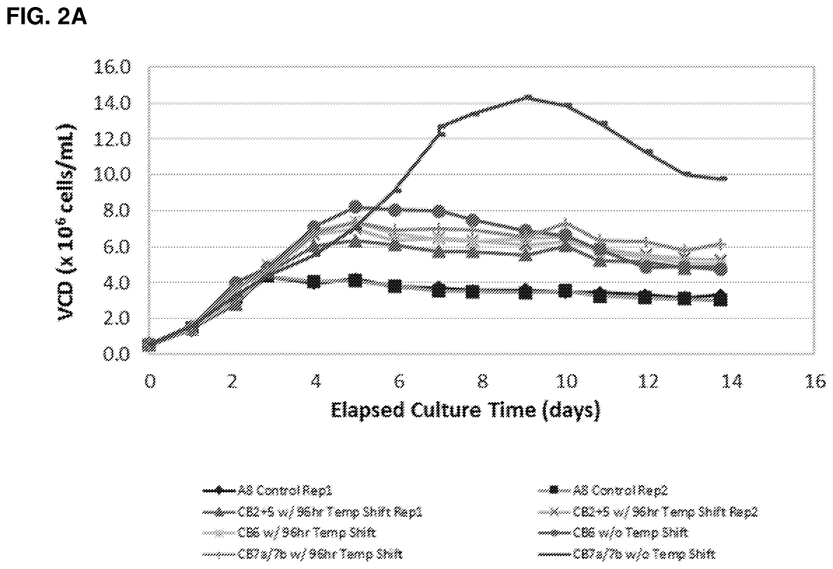

FIG. 2 displays the viable cell density (VCD) and cell viability data for Bioreactors ID Nos. 1-8. Addition of nutrient feeds promoted cell growth and resulted in overall higher IVCC for all conditions tested.

Control Process and Cell Boost 2+5 control bioreactors showed similar cell culture performance, trending similar to historical data, i.e., average 1.6-fold increase in overall IVCC. Cell Boost 6 supplementation, with or without temperature shift, showed similar cell growth as Cell Boost 2+5 supplemented bioreactors with temperature shift, while highest VCD (14×10⁶ cells/mL) was observed for bioreactor supplemented with Cell Boost 7a/7b, without temperature shift, a potential 2.7-fold increase in overall IVCC. Removing the temperature shift from Cell Boost 7a/7b reactors resulted in a 3.7 times higher peak VCD, demonstrating the potential to significantly improve cell growth by adjusting the temperature shift timing. With temperature shift implemented for bioreactor supplemented with Cell Boost 7a/7b, VCD growth was similar to other Cell Boost supplemented conditions. Delaying temperature shift prolonged the exponential growth phase of the cells, resulting in a higher peak VCD. In contrast, removing temperature shift from Cell Boost 6 bioreactors did not significantly change peak VCD, but instead led to a faster decline in viable cells. A lower harvest viability is known to be detrimental to product quality. Furthermore, temperature shift at 96 hours post inoculation did not affect cell viability decline rate in the Cell Boost 7a/7b supplemented bioreactor.

The use of nutrient supplement Efficient Feed C+ and Cell Boost 2+4 did not result in any significant increase in productivity (data not shown). The use of Cell Boost 2+5 and Cell Boost 6 increased productivity 2-fold (product titer). Removing the temperature shift from Cell Boost 6 bioreactors did not improve the productivity results.

The use of Cell Boost 7a+7b condition delivered the most surprising results. This specific feed supplementation increased total productivity by 6.5-fold, without temperature shift.

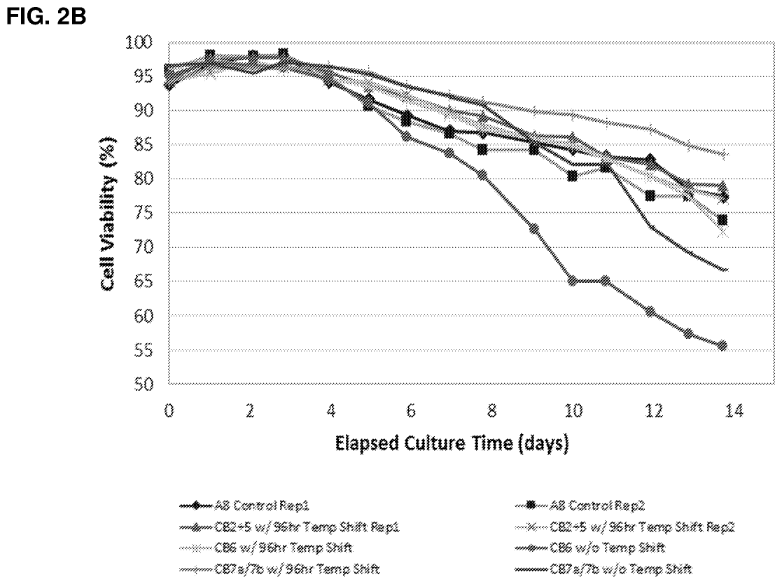

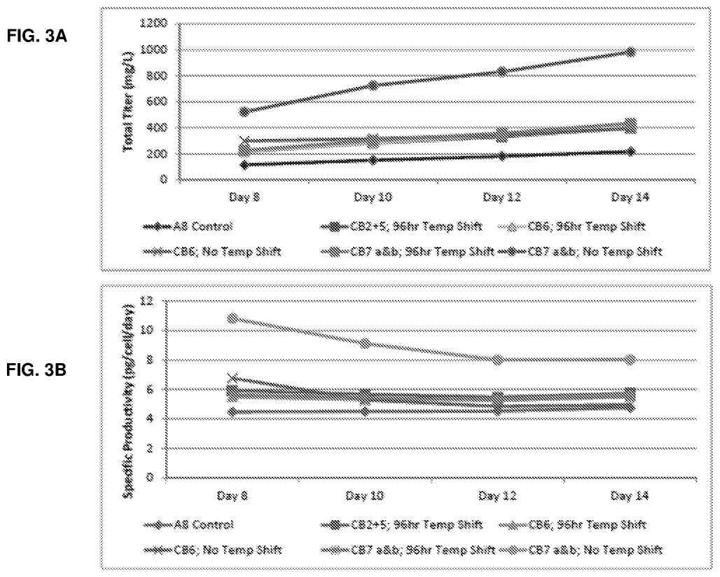

Specific asfotase alfa activity and productivity were evaluated. ProA bindable titer, specific productivity, total activity, and specific activity data for Bioreactor ID Nos. 1-8 are presented in FIG. 3 and FIG. 4.

All conditions supplemented with Cell Boost feed showed higher titer and specific productivity over Control Process. Increased cell densities did not have a detrimental effect on cell productivity. Specific productivity profiles did not drift significantly between conditions. Bioreactor supplemented with Cell Boost 7a/7b, without temperature shift, produced 981 mg/L of protein product by day 14 (˜5 times the titer of Control Process), while implementing a temperature shift under the Cell Boost 7a/7b combination fed conditions resulted in similar levels of protein titer as other Cell Boost supplemented conditions. This indicates that the volumetric titer range under Cell Boost 7a/7b fed condition could be further modulated by modifying temperature shift timing.

Specific productivity levels of bioreactors with temperature shift condition remained largely unchanged between day 8 and day 14. A gradual drop in specific productivity was seen for bioreactors without temperature shift. This effect could have been caused by lack of nutrients and/or insufficient sparging to support higher cell growth, as seen in Cell Boost supplemented bioreactors.

All Cell Boost supplemented bioreactors showed higher total volumetric activity than Control Process, consistent with the higher productivity levels. Cell Boost 6 and Cell Boost 7a/7b supplemented bioreactors, with temperature shift condition, showed similar total volumetric activity throughout the run (234 U/mL on day 14). However, Cell Boost 7a/7b supplementation without temperature shift condition showed a decrease in total volumetric activity between day 12 (229 U/mL) and day 14 (133 U/mL).

All conditions with temperature shift demonstrated similar specific activities as the Control Process, while those without temperature shift showed almost 60% lower specific activity than Control Process conditions, which is consistent with previous findings that temperature shift enhanced the specific activity. The reduction in volumetric activity for Cell Boost 7a/7b supplemented bioreactor, with temperature shift condition, was due to a reduction in specific activity in the bioreactor from day 12 (276 U/mg) to day 14 (136 U/mg).

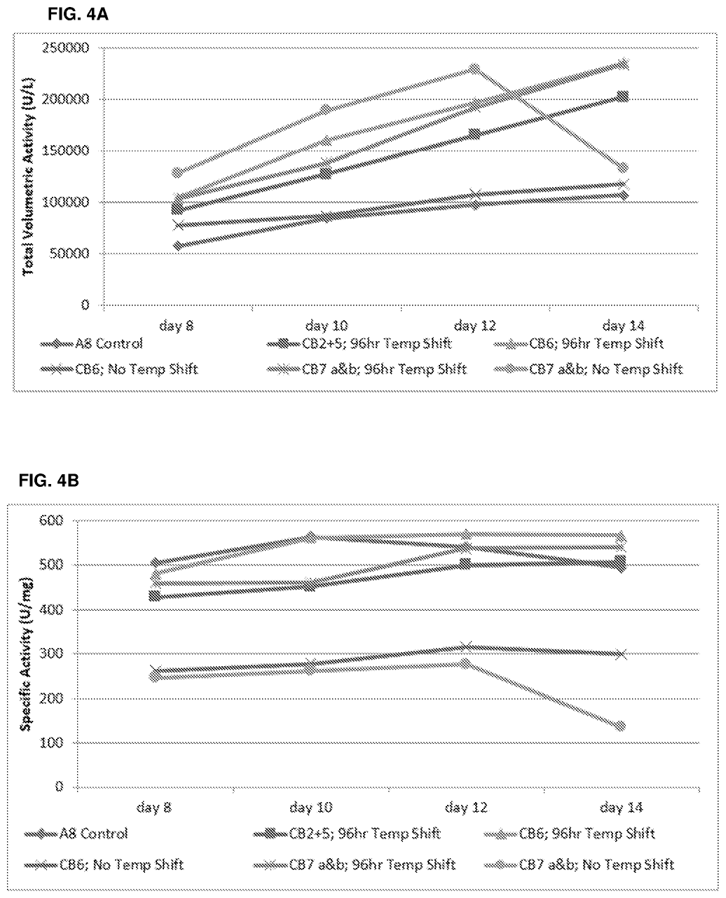

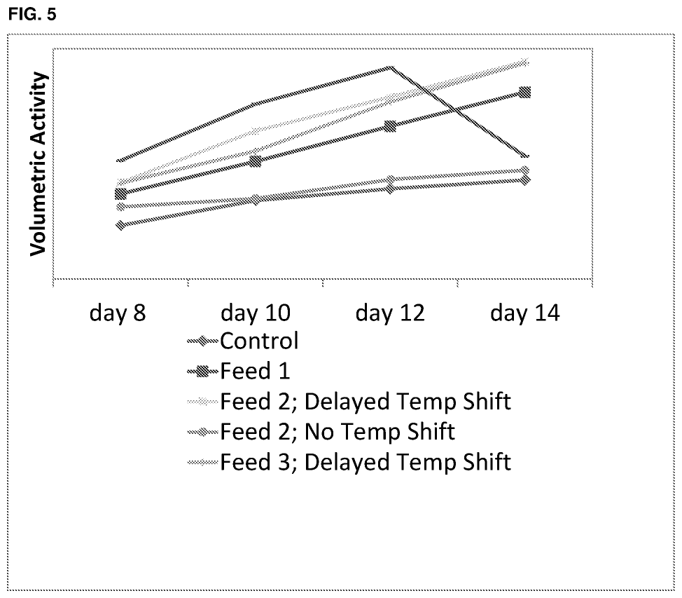

Protein activity is important for manufacturing asfotase alfa and other glycoproteins. Total volumetric activity was boosted by additional feed supplementation, as a result of increased IVCC. Cell Boost 2+5 conditions provided a 1.8-fold increase in total activity, while Cell Boost 6 and Cell Boost 7a/7b conditions resulted in 2.2 times higher total activity, compared to Control Process. Specific activity profile did not change across different feed supplements. However, a delay in temperature shift hampered specific activity levels significantly. These results highlight the importance of adjusting temperature shift timing to ensure the highest percentage of active product. See FIG. 5 and FIG. 6.

### Example 4: Determination of Aggregates By SEC-HPLC

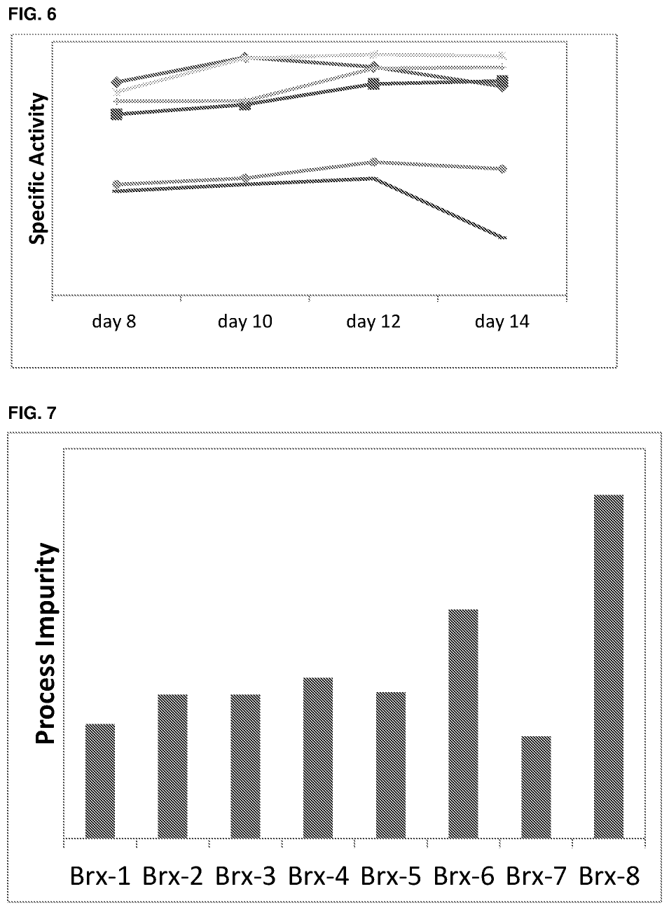

SEC-HPLC data is presented in Table 3 and FIG. 7. Increase in overall IVCC and productivity for alternative feeding conditions (with temperature shift) did not significantly change process impurity at harvest. Removing temperature shift from the conditions, however, resulted in impurity levels as high as 2.6 times the control, highlighting the importance the temperature shift to control process impurity levels. In general, higher aggregates were observed from day 10 to day 14 for all tested conditions. Cell Boost 7a/7b supplemented bioreactor, with a 96 hour temperature shift, demonstrated similar aggregate levels (4.2%) to the Control Process bioreactors (4.1 and 4.2% on day 10), despite its almost double productivity on day 14. With the same feeding strategy, temperature shift-lacking conditions resulted in higher aggregate than their corresponding temperature-shifted conditions.

TABLE 3
SEC Data

| | Bioreactor | SEC (Aggregate %) | |
|---|---|---|---|
| Condition | ID | Day 10 | Day 14 |
| Control Process | 1 | 4.2 | 4.7 |
| | 2 | 4.1 | 5.9 |
| CB2 + 5; 96 hr Temp Shift | 3 | 4.8 | 5.9 |
| | 4 | 5.0 | 6.6 |
| CB6; 96 hr Temp Shift | 5 | 5.4 | 6.0 |
| CB6; No Temp Shift | 6 | 8.0 | 9.4 |
| CB7a & b; 96 hr Temp Shift | 7 | 4.2 | 4.2 |
| CB7a & b; No Temp Shift | 8 | 10.0 | 14.1 |

### Example 5: Determination of Total Sialic Acid Content (TSAC)

Protein sialylation is a critical protein product attribute, and impacts protein half-life under physiological conditions. Production processes must be chosen in order to tightly control sialylation levels within acceptable limits.

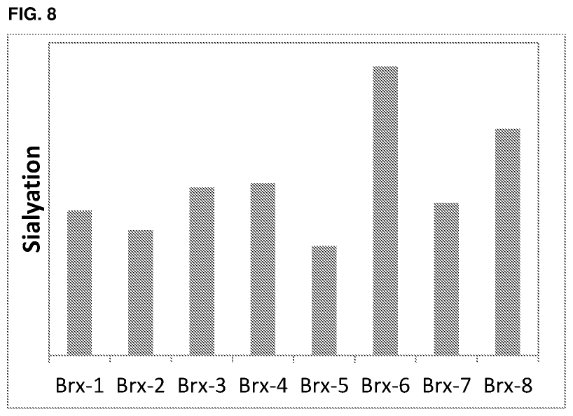

TSAC data is presented in Table 4 and FIG. 8. All alternative nutrient supplement conditions with a temperature shift reported acceptable sialylation levels at harvest. Bioreactors without temperature shift led to significantly higher TSAC than those with temperature shift implemented. Cell Boost 7a/7b supplemented bioreactor, with temperature shift condition, showed a TSAC of 3.9 mole/mole, very similar to average Control Process TSAC values (3.4) on day 14. These results confirm the importance of the temperature shift in the production process.

TABLE 4
TSAC Data

| | |
|---|---|
| Bioreactor | TSAC |

| Condition | ID | Day 10 | Day 14 |
|---|---|---|---|
| Control Process | 1 | 4.9 | 3.7 |
| | 2 | 3.0 | 3.2 |
| CB2 + 5; 96 hr Temp Shift | 3 | 4.8 | 4.3 |
| | 4 | 5.0 | 4.4 |
| CB6; 96 hr Temp Shift | 5 | 4.0 | 2.8 |
| CB6; No Temp Shift | 6 | 7.2 | 7.4 |
| CB7a & b; 96 hr Temp Shift | 7 | 4.4 | 3.9 |
| CB7a & b; No Temp Shift | 8 | 6.0 | 5.8 |

The most promising condition (BD Select supplemented in culture medium, Cell Boost 7a/7b supplemented as feed, Zinc sulfate supplemented, and temperature shift implemented) led to volumetric activity 234 U/mL (×2 folder higher than Control Process) and comparable product quality attributes (TSAC and SEC) with respect to Control Process. In addition, the combination of Cell Boost 7a/7b demonstrated a great potential to increase volumetric productivity via enhancing cell growth.

The results suggest Cell Boost 7a+7b delivered the most promising results, showing an increase in productivity by 6.5-fold compared to the Control Process upstream process. Delaying temperature shift prolongs exponential growth phase of cells and promotes higher overall IVCC. The results also confirm the importance of temperature shift in this upstream production process in achieving desired product quality

### Example 6: Evaluation of Zinc Salts Culture Media Supplementation on Asfotase Alfa Production

Zinc sulfate will be added to the asfotase alfa culture medium, at least one day post inoculation, at concentrations ranging from 20 μM to 200 μM Zn²⁺ according to the conditions in Examples 1-5. Growth, activity, specific activity, productivity, aggregates, and sialylation will be evaluated at Day 10 and Day 14. Other physiologically acceptable and commercially available zinc salts will also be evaluated, including but not limited to sulfide, bromide, chloride, fluoride, iodide, phosphate, selenide, nitrate, etc.

### Example 7: Evaluation of Temperature Shift Timing

The specific timing of the temperature shift will be further evaluated in conjuction with the nutrient supplementation in accordance with the conditions in Examples 1-6 for asfotase alfa production in CHO cells. Growth, activity, specific activity, productivity, aggregates, and sialylation will be evaluated at Day 10 and Day 14. Results will achieve the highest active product levels while preserving product quality and acceptable release parameters.

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits of the instant disclosure will be apparent to those skilled in the art without departing from the scope and spirit of the claimed invention. Although the disclosure has been described in connection with specific embodiments, it will be understood that it is capable of further modifications and that the invention as claimed should not be unduly limited to such specific embodiments.

1. A method of producing recombinant alkaline phosphatase comprising:
(i) inoculating Chinese Hamster Ovary (CHO) cells expressing the recombinant alkaline phosphatase in culture medium;
(ii) culturing the CHO cells in the culture medium at a temperature of from about 36° C. to about 38° C.;
(iii) adding:
(a) a combination of nutrient supplements to the cell culture of (ii) at least one day after inoculation, the combination comprising
a first animal-derived component-free (ADCF) nutrient supplement comprising one or more amino acids, vitamins, salts, trace elements, poloxamer and glucose, wherein the first ADCF nutrient supplement does not comprise hypoxanthine, thymidine, insulin, L-glutamine, growth factors, peptides, proteins, hydrolysates, phenol red, and 2-mercaptoethanol; and
a second ADCF nutrient supplement comprising one or more amino acids, wherein the second ADCF nutrient supplement lacks hypoxanthine, thymidine, insulin, L-glutamine, growth factors, peptides, proteins, hydrolysates, phenol red, 2-mercaptoethanol, and poloxamer; or
(b) at least one nutrient supplement to the cell culture, and adding from about 20 μM to about 200 μM Zn²⁺;
(iv) decreasing the temperature of the cell culture of (iii) to about 30° C. about 80 hours to about 150 hours after the inoculation; and
(v) isolating the recombinant alkaline phosphatase from the cell culture of (iv) by at least one chromatography step.

2. The method of claim 1, wherein the culture medium is selected from the group consisting of EX-CELL® 302 Serum-Free Medium; CD DG44 Medium; BD Select™ Medium; SFM4CHO Medium, or a combination thereof.

3. The method of claim 1, wherein the culture medium comprises a combination of SFM4CHO Medium and BD Select™ Medium.

4. The method of claim 3, wherein the culture medium comprises a combination of SFM4CHO Medium and BD Select™ Medium at a ratio selected from 90/10, 80/20, 75/25, 70/30, 60/40, or 50/50; particularly at a ratio of 75/25.

5. The method of claim 1, wherein the combination of the first and second nutrient supplements is added:
(a) in a bolus;
(b) over a period of time ranging from 1 minute to 2 hours;
(c) 1 to 3 days after inoculation;
(d) at more than 2 different times;
(e) at 2 to 6 different times;
(f) at 4 to 5 different times;
(g) 1 to 3 days after inoculation and 3 to 5 days after inoculation;
(h) 1 to 3 days after inoculation, 3 to 5 days after inoculation, 5 to 7 days after inoculation, 7 to 9 days after inoculation, and 9 to 11 days after inoculation; or
(i) about 2 days after inoculation and about 4 days after inoculation, about 6 days after inoculation, about 8 days after inoculation, and about 10 days after inoculation.

6-13. (canceled)

14. The method of claim 1, wherein:
(a) each addition of the first nutrient supplement is added at a concentration of about 0.5% to about 4% (w/v) of the culture medium;
(b) each addition of the second nutrient supplement is added at a concentration of 0.05% to 0.8% (w/v) of the culture medium;
(c) the total addition of the first nutrient supplement is added at a concentration of 5% to 20% (w/v) of the culture medium; and/or
(d) the total addition of the second nutrient supplement is added at a concentration of 0.5% to 2% (w/v) of the culture medium.

15-17. (canceled)

18. The method of claim, wherein:
(a) the total addition of the first nutrient supplement is added at a concentration of 12% (w/v) of the culture medium;
(b) the total addition of the second nutrient supplement is added at a concentration of 1.2% (w/v) of the culture medium;
(c) each addition of the first nutrient supplement is added at a concentration of 2% (w/v) of the culture medium; and/or
(d) each addition of the second nutrient supplement is added at a concentration of 0.2% (w/v) of the culture medium.

19-21. (canceled)

22. The method of claim 1, wherein the first nutrient supplement is CELL BOOST™ 7a, and the second nutrient supplement is CELL BOOST™ 7b.

23. The method of claim 1, wherein the temperature decrease of (iv) is:
(a) about 80 hours to 120 hours after the inoculation;
(b) about 90 hours to 100 hours after the inoculation; or
(c) about 96 hours after the inoculation.

24-25. (canceled)

26. The method of claim 1, further comprising:
(a) providing zinc in the culture medium at a concentration of from at least about 20 μM to about 200 μM;
(b) providing zinc in the culture medium at a concentration of about 30, 50, 60, 90, 150, or 200 μM; or
(c) providing zinc in the culture medium at a concentration of about 90 μM.

27-28. (canceled)

29. The method of claim 1, wherein:
(a) step (v) occurs 10 or 14 days after inoculation;
(b) step (v) comprises at least one of harvest clarification, ultrafiltration, diafiltration, viral inactivation, affinity capture, and combinations thereof; and/or
(c) step (ii) comprises culturing the CHO cells at a temperature from about 36.5° C. to about 37.5° C.

30-31. (canceled)

32. The method of claim 1, further comprising measuring recombinant alkaline phosphatase activity and/or determining an integral of viable cell concentration (IVCC).

33. The method of claim 32, wherein measuring the recombinant alkaline phosphatase activity comprises at least one of a pNPP-based alkaline phosphatase enzymatic assay and an inorganic pyrophosphate (PPi) hydrolysis assay.

34. The method of claim 33, wherein the recombinant alkaline phosphatase exhibits an increase in at least one of K_{cat} and Kₘ values in the inorganic pyrophosphate (PPi) hydrolysis assay.

35. (canceled)

36. The method of claim 32, wherein the IVCC is increased by from about 3.0-fold to about 6.5-fold as compared to IVCC of a recombinant alkaline phosphatase produced in the absence of steps (iii) and (iv).

37. The method of claim 1, wherein the recombinant alkaline phosphatase comprises the structure of W-sALP-X-Fc-Y-Dₙ-Z, wherein
W is absent or is an amino acid sequence of at least one amino acid;
X is absent or is an amino acid sequence of at least one amino acid;
Y is absent or is an amino acid sequence of at least one amino acid;
Z is absent or is an amino acid sequence of at least one amino acid;
Fc is a fragment crystallizable region;
Dₙ is a poly-aspartate, poly-glutamate, or combination thereof, wherein n=10 or 16; and
said sALP is a soluble alkaline phosphatase.

38. The method of claim 37, wherein at least one of:
(a) said sALP comprises an active anchored form of alkaline phosphatase (ALP) without C-terminal glycolipid anchor (GPI);
(b) said alkaline phosphatase (ALP) is tissue-non-specific alkaline phosphatase (TNALP);
(c) said sALP is encoded by a polynucleotide encoding a polypeptide comprising the sequence as set forth in L1-S485 of SEQ ID NO:1;
(d) said sALP comprises the sequence as set forth in L1-S485 of SEQ ID NO:1;
(e) said sALP is capable of catalyzing the cleavage of inorganic pyrophosphate (PPi);
(f) n=10;
(g) W and Z are absent from said polypeptide;
(h) said Fc comprises a CH₂ domain, a CH₃ domain and a hinge region;
(i) said Fc is a constant domain of an immunoglobulin selected from the group consisting of IgG-1, IgG-2, IgG-3, IgG-3 and IgG-4;
(j) said Fc is a constant domain of an immunoglobulin IgG-1;
(k) said Fc comprises the sequence as set forth in D488-K714 of SEQ ID NO:1;
(l) the recombinant alkaline phosphatase is encoded by a polynucleotide encoding a polypeptide comprising the sequence as set forth in SEQ ID NO:1;
(m) the recombinant alkaline phosphatase comprises the sequence as set forth in SEQ ID NO:1 and is a dimer thereof; or
(n) the recombinant alkaline phosphatase is encoded by a first polynucleotide which hybridizes under high stringency conditions to a second polynucleotide comparing the sequence completely complementary to a third polynucleotide encoding a polypeptide comprising the sequence as set forth in SEQ ID NO:1, wherein said high stringency conditions comprise: pre-hybridization and hybridization in 6×SSC, 5× Denhardt's reagent, 0.5% SDS and 100 mg/ml of denatured fragmented salmon sperm DNA at 68° C.; and washes in 2×SSC and 0.5% SDS at room temperature for 10 minutes; in 2×SSC and 0.1% SDS at room temperature for 10 minutes; and in 0.1×SSC and 0.5% SDS at 65° C. three times for 5 minutes.

39-51. (canceled)

52. The method of claim 38, wherein the recombinant alkaline phosphatase comprises an amino acid sequence having at least 90% sequence identity to SEQ ID NO:1.

53. The method of claim 52, wherein the recombinant alkaline phosphatase comprises the amino acid sequence as set forth in SEQ ID NO:1.

54-106. (canceled)

107. The method of claim 29, wherein step (ii) comprises culturing the CHO cells at a temperature of about 37° C.


SEQUENCE LISTING (1 sequence)

SEQ ID NO: 1 - protein, 726 aa, Homo sapiens
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDIDDDDDDDDDD